SEQUENCE LISTING (24 sequences)

SEQ ID NO: 1 - protein, 79 aa, Homo sapiens
  MISC_FEATURE (2)..(2): Applicants herein disclose Thr and Ser sequence variants at this position
GXHSXXPRPAAVPVPXXXQPXPAHPVLSFLXPSWDXVSAFYSLPLAPLSPTSVXISPVSVGRGXDPDAHVAVXLSRYEG

SEQ ID NO: 2 - protein, 419 aa, Homo sapiens
  MISC_FEATURE (342)..(342): Applicants herein disclose Thr and Ser sequence variants at this position
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLRRTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGXHSXXPRPAAVPVPXXXQPXPAHPVLSFLXPSWDXVSAFYSLPLAPLDPTSVXISPVSVGRGXDPDAHVAVXLSRYEG

SEQ ID NO: 3 - protein, 79 aa, artificial sequence
  ECDIIIA domain non-binding mutant (Arg to Ile mutation at residue 31)
GTHSLPPRPAAVPVPLRMQPGPAHPVLSFLIPSWDLVSAFYSLPLAPLSPTSVPISPVSVGRGPDPDAHVAVDLSRYEG

SEQ ID NO: 4 - protein, 419 aa, artificial sequence
  Herstatin; receptor non-binding mutant (Arg to Ile mutation at residue 371)
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRMQPGPAHPVLSFLIPSWDLVSAFYSLPLAPLSPTSVPISPVSVGRGPDPDAHVAVDLSRYEG

SEQ ID NO: 5 - DNA, 5616 bp, Homo sapiens
  CDS (247)..(3879): HER-1 coding sequence
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 6 - protein, 1210 aa, Homo sapiens [sequence omitted]

SEQ ID NO: 7 - DNA, 4815 bp, Homo sapiens
  CDS (247)..(3078): delta EGFR coding sequence [represents in-frame deletion of 801 bp (275-1027) of the ECD of EGFR corresponding to exons 2-7 of the gene]
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagtggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 8 - protein, 943 aa, Homo sapiens
MRPSGTAGAVDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 9 - DNA, 4530 bp, Homo sapiens
  CDS (151)..(3918): HER-2 coding sequence
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 10 - protein, 1255 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 11 - DNA, 4975 bp, Homo sapiens
  CDS (199)..(4227)
ctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggggggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggggtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctc

SEQ ID NO: 12 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMGGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGGSCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 13 - DNA, 4975 bp, Homo sapiens
  CDS (199)..(4227): HER-3 mutant coding sequence
ctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggggtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctc

SEQ ID NO: 14 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGGSCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 15 - DNA, 5484 bp, Homo sapiens
  CDS (34)..(3960): HER-4 coding sequence
aattgtcagcacgggatctgagacttccaaaaaatgaagccggcgacaggactttgggtctgggtgagccttctcgtggcggcggggaccgtccagcccagcgattctcagtcagtgtgtgcaggaacggagaataaactgagctctctctctgacctggaacagcagtaccgagccttgcgcaagtactatgaaaactgtgaggttgtcatgggcaacctggagataaccagcattgagcacaaccgggacctctccttcctgcggtctgttcgagaagtcacaggctacgtgttagtggctcttaatcagtttcgttacctgcctctggagaatttacgcattattcgtgggacaaaactttatgaggatcgatatgccttggcaatatttttaaactacagaaaagatggaaactttggacttcaagaacttggattaaagaacttgacagaaatcctaaatggtggagtctatgtagaccagaacaaattcctttgttatgcagacaccattcattggcaagatattgttcggaacccatggccttccaacttgactcttgtgtcaacaaatggtagttcaggatgtggacgttgccataagtcctgtactggccgttgctggggacccacagaaaatcattgccagactttgacaaggacggtgtgtgcagaacaatgtgacggcagatgctacggaccttacgtcagtgactgctgccatcgagaatgtgctggaggctgctcaggacctaaggacacagactgctttgcctgcatgaatttcaatgacagtggagcatgtgttactcagtgtccccaaacctttgtctacaatccaaccacctttcaactggagcacaatttcaatgcaaagtacacatatggagcattctgtgtcaagaaatgtccacataactttgtggtagattccagttcttgtgtgcgtgcctgccctagttccaagatggaagtagaagaaaatgggattaaaatgtgtaaaccttgcactgacatttgcccaaaagcttgtgatggcattggcacaggatcattgatgtcagctcagactgtggattccagtaacattgacaaattcataaactgtaccaagatcaatgggaatttgatctttctagtcactggtattcatggggacccttacaatgcaattgaagccatagacccagagaaactgaacgtctttcggacagtcagagagataacaggtttcctgaacatacagtcatggccaccaaacatgactgacttcagtgttttttctaacctggtgaccattggtggaagagtactctatagtggcctgtccttgcttatcctcaagcaacagggcatcacctctctacagttccagtccctgaaggaaatcagcgcaggaaacatctatattactgacaacagcaacctgtgttattatcataccattaactggacaacactcttcagcacaatcaaccagagaatagtaatccgggacaacagaaaagctgaaaattgtactgctgaaggaatggtgtgcaaccatctgtgttccagtgatggctgttggggacctgggccagaccaatgtctgtcgtgtcgccgcttcagtagaggaaggatctgcatagagtcttgtaacctctatgatggtgaatttcgggagtttgagaatggctccatctgtgtggagtgtgacccccagtgtgagaagatggaagatggcctcctcacatgccatggaccgggtcctgacaactgtacaaagtgctctcattttaaagatggcccaaactgtgtggaaaaatgtccagatggcttacagggggcaaacagtttcattttcaagtatgctgatccagatcgggagtgccacccatgccatccaaactgcacccaagggtgtaacggtcccactagtcatgactgcatttactacccatggacgggccattccactttaccacaacatgctagaactcccctgattgcagctggagtaattggtgggctcttcattctggtcattgtgggtctgacatttgctgtttatgttagaaggaagagcatcaaaaagaaaagagccttgagaagattcttggaaacagagttggtggaaccattaactcccagtggcacagcacccaatcaagctcaacttcgtattttgaaagaaactgagctgaagagggtaaaagtccttggctcaggtgcttttggaacggtttataaaggtatttgggtacctgaaggagaaactgtgaagattcctgtggctattaagattcttaatgagacaactggtcccaaggcaaatgtggagttcatggatgaagctctgatcatggcaagtatggatcatccacacctagtccggttgctgggtgtgtgtctgagcccaaccatccagctggttactcaacttatgccccatggctgcctgttggagtatgtccacgagcacaaggataacattggatcacaactgctgcttaactggtgtgtccagatagctaagggaatgatgtacctggaagaaagacgactcgttcatcgggatttggcagcccgtaatgtcttagtgaaatctccaaaccatgtgaaaatcacagattttgggctagccagactcttggaaggagatgaaaaagagtacaatgctgatggaggaaagatgccaattaaatggatggctctggagtgtatacattacaggaaattcacccatcagagtgacgtttggagctatggagttactatatgggaactgatgacctttggaggaaaaccctatgatggaattccaacgcgagaaatccctgatttattagagaaaggagaacgtttgcctcagcctcccatctgcactattgacgtttacatggtcatggtcaaatgttggatgattgatgctgacagtagacctaaatttaaggaactggctgctgagttttcaaggatggctcgagaccctcaaagatacctagttattcagggtgatgatcgtatgaagcttcccagtccaaatgacagcaagttctttcagaatctcttggatgaagaggatttggaagatatgatggatgctgaggagtacttggtccctcaggctttcaacatcccacctcccatctatacttccagagcaagaattgactcgaataggagtgaaattggacacagccctcctcctgcctacacccccatgtcaggaaaccagtttgtataccgagatggaggttttgctgctgaacaaggagtgtctgtgccctacagagccccaactagcacaattccagaagctcctgtggcacagggtgctactgctgagatttttgatgactcctgctgtaatggcaccctacgcaagccagtggcaccccatgtccaagaggacagtagcacccagaggtacagtgctgaccccaccgtgtttgccccagaacggagcccacgaggagagctggatgaggaaggttacatgactcctatgcgagacaaacccaaacaagaatacctgaatccagtggaggagaacccttttgtttctcggagaaaaaatggagaccttcaagcattggataatcccgaatatcacaatgcatccaatggtccacccaaggccgaggatgagtatgtgaatgagccactgtacctcaacacctttgccaacaccttgggaaaagctgagtacctgaagaacaacatactgtcaatgccagagaaggccaagaaagcgtttgacaaccctgactactggaaccacagcctgccacctcggagcacccttcagcacccagactacctgcaggagtacagcacaaaatatttttataaacagaatgggcggatccggcctattgtggcagagaatcctgaatacctctctgagttctccctgaagccaggcactgtgctgccgcctccaccttacagacaccggaatactgtggtgtaagctcagttgtggttttttaggtggagagacacacctgctccaatttccccacccccctctctttctctggtggtcttccttctaccccaaggccagtagttttgacacttcccagtggaagatacagagatgcaatgatagttatgtgcttacctaacttgaacattagagggaaagactgaaagagaaagataggaggaaccacaatgtttcttcatttctctgcatgggttggtcaggagaatgaaacagctagagaaggaccagaaaatgtaaggcaatgctgcctactatcaaactagctgtcactttttttctttttctttttctttctttgtttctttcttcctcttctttttttttttttttttaaagcagatggttgaaacacccatgctatctgttcctatctgcaggaactgatgtgtgcatatttagcatccctggaaatcataataaagtttccattagaacaaaagaataacattttctataacatatgatagtgtctgaaattgagaatccagtttctttccccagcagtttctgtcctagcaagtaagaatggccaactcaactttcataatttaaaaatctccattaaagttataactagtaattatgttttcaacactttttggtttttttcattttgttttgctctgaccgattcctttatatttgctcccctatttttggctttaatttctaattgcaaagatgtttacatcaaagcttcttcacagaatttaagcaagaaatattttaatatagtgaaatggccactactttaagtatacaatctttaaaataagaaagggaggctaatatttttcatgctatcaaattatcttcaccctcatcctttacatttttcaacatttttttttctccataaatgacactacttgataggccgttggttgtctgaagagtagaagggaaactaagagacagttctctgtggttcaggaaaactactgatactttcaggggtggcccaatgagggaatccattgaactggaagaaacacactggattgggtatgtctacctggcagatactcagaaatgtagtttgcacttaagctgtaattttatttgttctttttctgaactccattttggattttgaatcaagcaatatggaagcaaccagcaaattaactaatttaagtacatttttaaaaaaagagctaagataaagactgtggaaatgccaaaccaagcaaattaggaaccttgcaacggtatccagggactatgatgagaggccagcacattatcttcatatgtcacctttgctacgcaaggaaatttgttcagttcgtatacttcgtaagaaggaatgcgagtaaggattggcttgaattccatggaatttctagtatgagactatttatatgaagtagaaggtaactctttgcacataaattggtataataaaaagaaaaacacaaacattcaaagcttagggataggtccttgggtcaaaagttgtaaataaatgtgaaacatcttctc

SEQ ID NO: 16 - protein, 1308 aa, Homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 17 - DNA, 4989 bp, Homo sapiens
  CDS (46)..(4149): IGF-1R coding sequence
ttttttttttttttgagaaagggaatttcatcccaaataaaaggaatgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggaggcaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctgatccttggatcctgaatctgtgcaaacagtaacgtgtgcgcacgcgcagcggggtggggggggagagagagttttaacaatccattcacaagcctcctgtacctcagtggatcttcagttctgcccttgctgcccgcgggagacagcttctctgcagtaaaacacatttgggatgttccttttttcaatatgcaagcagctttttattccctgcccaaacccttaactgacatgggcctttaagaaccttaatgacaacacttaatagcaacagagcacttgagaaccagtctcctcactctgtccctgtccttccctgttctccctttctctctcctctctgcttcataacggaaaaataattgccacaagtccagctgggaagccctttttatcagtttgaggaagtggctgtccctgtggccccatccaaccactgtacacacccgcctgacaccgtgggtcattacaaaaaaacacgtggagatggaaatttttacctttatctttcacctttctagggacatgaaatttacaaagggccatcgttcatccaaggctgttaccattttaacgctgcctaattttgccaaaatcctgaactttctccctcatcggcccggcgctgattcctcgtgtccggaggcatgggtgagcatggcagctggttgctccatttgagagacacgctggcgacacactccgtccatccgactgcccctgctgtgctgctcaaggccacaggcacacaggtctcattgcttctgactagattattatttgggggaactggacacaataggtctttctctcagtgaaggtggggagaagctgaaccggc

SEQ ID NO: 18 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 19 - DNA, 22 bp, artificial sequence
  exon 8 primer (3′ PCR amplification primer; see EXAMPLE I)
aacacagcggtgtgagaagtgc

SEQ ID NO: 20 - DNA, 28 bp, artificial sequence
  intron 9 primer (5′ PCR amplification primer; see EXAMPLE I)
gtatcggtagttcatttcctttggttgc

SEQ ID NO: 21 - DNA, 20 bp, artificial sequence
  3′ PCR amplification primer for rat intron 8 region
ctacctgtctacggaagtgg

SEQ ID NO: 22 - DNA, 20 bp, artificial sequence
  5′ PCR amplification primer for rat intron 8 region
ttccgggcagaaatgccagg

SEQ ID NO: 23 - protein, 419 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRMQPGPAHPVLSFLRPSWDLVSAFYSLPLAPLSPTSVPISPVSVGRGPDPDAHVAVDLSRYEG

SEQ ID NO: 24 - protein, 79 aa, Homo sapiens
GTHSLPPRPAAVPVPLRMQPGPAHPVLSFLRPSWDLVSAFYSLPLAPLSPTSVPISPVSVGRGPDPDAHVAVDLSRYEG

(19) United States
(12) Patent Application Publication
(10) Pub. No.: US 2005/0272637 A1
(43) Pub. Date: Dec. 8, 2005

(54) Compositions and methods for modulating signaling mediated by IGF-1 receptor and erbB receptors

(75) Inventors: Gail M. Clinton, Portland, OR (US); Lara Shamieh, Portland, OR (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Oregon Health & Science University, Portland, OR (US)

(21) Appl. No.: 11/113,202

(22) Filed: Apr. 22, 2005

Related U.S. Application Data
(60) Provisional application No. 60/590,473, filed on Jul. 23, 2004.
(60) Provisional application No. 60/564,893, filed on Apr. 22, 2004.

(51) Int. Cl. A61K 38/17

(52) U.S. Cl. 514/2

(57) ABSTRACT

The binding interactions between herstatin, or the intron 8-encoded receptor binding domain (RBD Int8) thereof, and several receptors were analyzed. According to aspects of the present invention, herstatin and the intron 8-encoded domain bind with high affinity (e.g., nM concentrations) to all four of the ErbB receptors: EGFR (HER-1, erbB-1); HER-2 (erbB-2); HER-3 (erbB-3); and HER-4 (erbB-4), as well as to ΔEGFR and the IGF-1 receptor, and such binding has utility to modulate signaling mediated by these receptors. Herstatin inhibited target receptor-mediated activation of intracellular signaling pathways ((e.g., PI3/Akt, IRS-2, etc., pathways) that are important in cell survival, and further inhibited target receptor-mediated (e.g., IGF-1/IGF-1R-mediated) survival of cancer cells. Aspects of the present invention thus provide methods and compositions for the treatment of cancer, including cancer refractory to other erbB-based agents, and of other conditions and disorders characterized by target receptor expression, over-expression, signaling, and/or aberrant signaling. Additional aspects provide methods of targeted drug delivery.

### CROSS-REFERENCE TO RELATED APPLICATIONS

This application claims the benefit of priority to U.S. Provisional Patent Application Ser. No. 60/590,473, filed 23 Jul. 2004, and entitled COMPOSITIONS AND METHODS FOR TREATING CANCER BY MODULATING IGF-1 RECEPTOR AND ERBB RECEPTORS, to U.S. Provisional Patent Application Ser. No. 60/564,893, filed 22 Apr. 2004, of same title, and to PCT application, filed 22 Apr. 2005, of same title, all of which are incorporated by reference herein in their entirety.

### STATEMENT REGARDING FEDERALLY-SPONSORED RESEARCH

This work was partially funded by NIH grant number CA83503, and the United States government has, therefore, certain rights to the present invention.

### FIELD OF THE INVENTION

This invention relates generally to signaling through IGF-1 receptors and through ErbB family member receptors, and more specifically to novel methods and compositions for modulating intracellular signaling mediated by IGF-1 receptor and by ErbB family receptors, for cell targeting, and for the treatment of cancer and other target receptor-mediated conditions and disorders.

### BACKGROUND

The ErbB receptor family consists of four receptor tyrosine kinases: EGFR (HER-1, erbB-1), HER-2 (erbB-2), HER-3 (erbB-3) and HER-4 (erbB-4). Aberrant expression of ErbB receptors by mutational activation, receptor overexpression, and tumor production of ligands contributes to the development and maintenance of a variety of human cancers (e.g., Olayioye et al., Embo J., 19:3159-67, 2000).

The ErbB receptors, with one exception, are activated by several ligands with an EGF core domain (EGF-related growth factors). HER-2 receptor, the exception, is recruited as a preferred dimer partner with other ligand-binding erbB receptors (Id). The eleven mammalian EGF-like ligands are all agonists, whereas Drosophila has the ligand ‘Argos’ that inhibits activation of the EGFR (Dougall et al., Oncogene 9:2109-23, 1994; Hynes & Stem, Biochim. Biophys. Acta 1198:165-84, 1994; Tzahar & Yarden, Biochim. Biophys. Acta 1377:25-37, 1998).

Although the HER-2 receptor does not directly bind EGF-like ligands, a secreted product of an HER-2 alternative transcript, herstatin, binds with high affinity (K_{D}≅14 nM) to the ectodomains of HER-2 and the EGF receptor (EGFR). Herstatin consists of a segment of the HER-2 ectodomain (340 amino acids that are identical to the N-terminal subdomains I and II), followed by 79 amino acids encoded by intron 8 of the HER-2 gene that function as a receptor binding domain (RBD) (Doherty et al., Proc. Natl. Acad. Sci. USA 96:10869-74, 1999). Herstatin blocks homomeric and heteromeric ErbB receptor interactions, inhibits activation of the P13K/Akt pathway initiated by EGF, TGFα, and Heregulin, causes growth arrest, and has substantial utility as an anti-cancer agent (Id, and see, e.g., Azios et al., Oncogene 20:5199-209, 2001; Jhabvala-Romero et al., Oncogene 22:8178-86, 2003; and Justman & Clinton, J. Biol. Chem. 277:20618-24, 2002).

Anti-erbB receptor antibody agents, such as the HER-2-specific antibody rhuMAb4D5 (HERCEPTIN™) have been approved for cancer therapy. Significantly, however, tumor cells may be inherently resistant, or gain resistance, to anti-erbB receptor therapies through activation of IGF-IR pathways (see, e.g., Chakravarti et al., Cancer Res. 62:200-7, 2002 (discussing IGF-1R-mediated resistance to AG1478, an EGFR tyrosine kinase inhibitor); Lu et al., J. Biol. Chem. 279:2856-65, 2004; Lu et al., J. Natl. Cancer Inst., 93:1852-7, 2001 (discussing IGF-1R-mediated resistance to Herceptin™, in the context of breast cancer); and Camp, 2005 (discussing IGF-1R-mediated resistance to Iressa, a small molecule EGFR inhibitor, in the context of breast and prostate cancer)). Activation of the IGF-I receptor (IGF-IR) by IGF-I promotes, inter alia, proliferation, survival, transformation, metastasis, and angiogenesis (see, e.g., Baserga, Hum. Pathol. 31:275-6, 2000; and Wang & Sun, Curr. Cancer Drug Targets 2:191-207, 2002), and signaling through both IGF-IR and EGF receptors is central to tumorigenesis.

There is, therefore, a pronounced need in the art not only to further investigate and characterize the interactions among the erbB family receptors, but to identify modulators of the signaling mediated by erbB receptors and IGF-1 receptors. There is a need in the art for a multi-functional inhibitor that simultaneously targets both the EGF and IGF-IR families. There is a pronounced need in the art to identify and develop modulators (e.g., inhibitors) of erbB receptors and of IGF-IR modulators as therapeutic agents (e.g., anti cancer agents). There is a need in the art to further assess the receptor-modulating utilities of herstatin and its intron 8-encoded RBD.

### SUMMARY OF THE INVENTION

According to particular aspects of the present invention, herstatin, and the intron 8-encoded domain thereof (referred to herein as “int8 RBD”), bind with high affinity (e.g., at nM concentrations) to: all four of the ErbB receptors EGFR (HER-1, erbB-1), HER-2 (erbB-2), HER-3 (erbB-3), and HER-4 (erbB-4); as well as to ΔEGFR and the IGF-1 receptor. Moreover, such target receptor binding has been shown and disclosed herein to have novel and substantial utility to modulate intracellular signaling mediated by these receptors.

Particular embodiments provide novel methods and compositions for the treatment of cancer and other conditions and disorders characterized by target receptor expression or over-expression, and/or target receptor mediated signaling or aberrant signaling.

Specific embodiments provide a method for treating cancer, comprising administering a therapeutically effective amount of herstatin, or of a variant thereof, that binds to the extracellular domain of a target receptor selected from the group consisting of: ΔEGFR; HER-3 (erbB-3); HER-4 (erbB-4), IGF-1R and combinations thereof, wherein the cancer cells express at least one of the target receptors. Alternatively, a therapeutically effective amount of a Int8 RBD polypeptide, or of a variant thereof, that binds to the extracellular domain of a target receptor selected from the group consisting of: ΔEGFR; HER-3 (erbB-3); HER-4 (erbB-4), IGF-1R and combinations thereof, is administered. The methods also encompass treatments where the cancer cells further express EGFR (HER-1, erbB-1), HER-2 (erbB-2) or both.

Further embodiments provide combination therapies, further comprising, administration of a therapeutically effective amount of: a receptor-specific antibody that binds to the extracellular domain of a target receptor selected from the group consisting of: EGFR (HER-1, erbB-1); ΔEGFR; HER-2 (erbB-2); HER-3 (erbB-3); HER-4 (erbB-4), and IGF-1R; or of a chemotherapeutic (e.g., anti-neoplastic) agent.

Additional embodiments provide pharmaceutical compositions for treating cancer and other conditions and disorders characterized by target receptor expression or over-expression, and/or target receptor-mediated signaling or aberrant signaling, comprising, along with a pharmaceutically acceptable diluent, carrier or excipient, herstatin, or a variant thereof, that binds to the extracellular domain of a target receptor selected from the group consisting of: ΔEGFR; HER-3 (erbB-3); HER-4 (erbB-4); IGF-1R and combinations thereof, wherein the cancer cells express at least one of the target receptors. Alternatively, the inventive compositions comprise, along with a pharmaceutically acceptable diluent, carrier or excipient, a Int8 RBD polypeptide, or a variant thereof, that binds to the extracellular domain of a target receptor selected from the group consisting of: ΔEGFR; HER-3 (erbB-3); HER-4 (erbB-4), IGF-1R and combinations thereof, wherein the cancer cells express at least one of the target receptors.

The compositions also have substantial utility in treatments where the target cells (e.g., cancer cells) further express EGFR (HER-1, erbB-1), HER-2 (erbB-2) or both.

Additional aspects provide novel methods of targeted drug delivery.

Specific embodiments provide methods for targeting a therapeutic agent to cancer cells, comprising attaching the therapeutic agent to herstatin, or to a variant thereof, that binds to the extracellular domain of a target receptor selected from the group consisting of: ΔEGFR; HER-3 (erbB-3); HER-4 (erbB-4); IGF-1R and combinations thereof, wherein the cancer cells express at least one of the target receptors. Alternatively, the therapeutic agent is attached to a Int8 RBD polypeptide, or a variant thereof, that binds to the extracellular domain of a target receptor selected from the group consisting of: ΔEGFR; HER-3 (erbB-3); HER-4 (erbB-4); IGF-r and combinations thereof, wherein the cancer cells express at least one of the target receptors.

The targeting methods encompass treatments wherein the cancer cells further express EGFR (HER-1, erbB-1), HER-2 (erbB-2) or both.

Preferably, for the above-described methods and compositions, the herstatin, or variant thereof, comprises a polypeptide selected from the group consisting of SEQ ID NO:2, or a fragment of SEQ ID NO:2 of about 80 to 419 contiguous residues in length, wherein the C-terminal 79 contiguous amino acids are present. Preferably, the herstatin, or variant thereof, further comprises at least one N-linked glycosylation site, and binds to the extracellular domain of EGF receptor with an affinity binding constant of at least about 10⁷ M⁻¹, or of at least about 10⁸ M⁻¹.

Preferably, for the above-described methods and compositions, the Int8 RBD polypeptide, or variant thereof, comprises a polypeptide selected from the group consisting of SEQ ID NO:1, or a fragment of SEQ ID NO:1 of about 50 to 79 contiguous residues in length. Preferably, the Int8 RBD polypeptide, or variant thereof binds to the extracellular domain of target receptor with an affinity binding constant of at least about 10⁷ M⁻¹, or of at least about 10⁸ M⁻¹.

Additional embodiments provide for a novel form of HER-3 (SEQ ID NO:14) that does not bind to herstatin or to Int8 RBD polypeptides, thus providing screening assays for cells having impaired responsiveness to herstatin or int8 RBD polypeptides.

### BRIEF DESCRIPTION OF THE DRAWINGS

FIG. 1A demonstrates that the RBD Int8 polypeptide, purified from bacteria and immobilized on Protein S Sepharose™ ‘pulled down’ IGF-IR from 3T3 cell extracts.

FIG. 1B illustrates a binding curve showing saturable binding by the RBD Int8 polypeptide that is specific for IGF-IR.

FIG. 1C illustrates the results of ELISA assays showing that herstatin, purified from transfected S2 insect cells, exhibited dose-dependent binding to IGF-1R at nM concentrations.

FIG. 1D illustrates binding curves showing that full-length herstatin exhibited saturation binding to IGF-IR 3T3 cells, demonstrating nM binding affinity.

FIGS. 2A and 2B show that herstatin prevented activation of IGF-1R by IGF-1 in MCF-7 cells. FIG. 2A shows a representative Western immunoblot of IGFI-R immunoprecipitation of IGF-I-treated MCF-7 and MCF-7/Hst cell lysates. FIG. 2B shows a graphical representation of two independent experiments of IGF-I-induced activation of the IGF-I receptor. The lower portion of FIG. 2A shows that herstatin not only prevented activation of IGF-1R by IGF-1 in MCF-7 cells, but also caused down-regulation of IGF-1R.

FIG. 3A shows, using ‘pull-down’ assays, that the herstatin RBD Int8 polypeptide bound in a specific manner to EGFR, HER-2, HER-4, IGF-1R and ΔEGFR, but did not bind to a mutant form of HER-3, to FGFR-3, or to mock-transfected cells.

FIG. 3B shows, using ELISA, that the Int8 polypeptide bound in a specific and dose-dependent manner to EGFR, HER-2, HER-4, and ΔEGFR, but not to a mutant form of HER-3, FGFR-3, or mock-transfected cells.

FIGS. 4A and 4B illustrate Western blot analyses of RBD Int8 polypeptide binding to different forms of HER-3: FIG. 4A shows lack of RBD Int8 polypeptide binding to a form of HER-3 having a single point mutation resulting in substitution of Glu for Gly in the ectodomain of HER-3 (Accession #: NM_{—}001982, nucleotide # 1877, and amino acid residue # 560).

FIG. 4B shows high-affinity binding by Int8 RBD polypeptide to endogenous HER-3 on MCF7 breast cancer cells, independent of ligand activation.

FIG. 4C shows binding of the Int8 RBD polypeptide to purified (wild-type) HER-3.

FIG. 5A illustrates a binding curve showing that the Int8 RBD polypeptide bound to HER-2-transfected Cos-7 cells (K_{D}=50±6 nM; open squares) and to EGFR-transfected Cos-7 cells (K_{D}=78±10 nM; filled squares) with binding affinities, assessed by comparative nonlinear regression analysis, that were not significantly different (P=0.40).

FIG. 5B illustrates a binding curve showing that the Int8 RBD polypeptide bound to the IGF-IR/3T3 cells with an affinity (K_{D}=70±21) that was not significantly different (P=0.96) from the affinity for HER-2/3T3 cells (K_{D}=66±16).

FIG. 6A illustrates binding curves showing a direct comparison of the binding of herstatin to 3T3/HER-2 and 3T3/IGF-IR cells.

FIG. 6B illustrates Cos-7 cell herstatin binding curves showing that the dissociation constant of herstatin for EGFR was similar to that of HER-2, and was unaffected by ligand occupation.

FIG. 6C is a saturation binding curve showing that herstatin exhibited saturation binding to endogenous receptors in A431 epidermoid carcinoma cells, which express very high levels of EGFR and low levels of other ErbB receptors.

FIGS. 7A and 7B show that while herstatin blocked intracellular signaling (MAPK phosphorylation) by Heregulin (the ligand for HER-3 and HER-4) in MCF-7 cells (FIG. 7A, right-most two time series in upper panel), it does not affect FGF signaling (MAPK phosphorylation) in MCF-7 cells (FIG. 7A, right-most two time series in lower panel), and did not inhibit IGF-1-mediated ERK phosphorylation in MCF-7 cells (FIG. 7B).

FIG. 7C shows that herstatin down-regulates HER-1, HER-3 and HER-4 receptors in MCF-7 cells.

FIG. 7D shows that herstatin blocks EGF/EGFR-mediated intracellular signaling (MAPK phosphorylation) in MCF-7 cells.

FIGS. 8A and 8B show that herstatin inhibited IGF-1/IGF-1R-mediated activation of the PI3/Akt pathway that is important in cell survival. FIG. 8A shows representative Western immunoblot showing IGF-I-induced Akt/PKB activation in MCF7 and MCF7/Hst cells. FIG. 8B shows the graphical representation of 3 separate experiments, according to FIG. 8A.

FIG. 9 shows the effect of herstatin-expression on the expression levels of various signaling proteins. Herstatin expression in MCF7 breast carcinoma cells down-regulated IGF-1R, IRS-1, IRS-2 (also important in cell survival), and pKB/Akt expression, but MAPK expression was unaffected. Herstatin expression also induced expression of the p66 isoform of Shc, which is not detectable by Western Blot in parental MCF7 cells.

FIGS. 10A and 10B show the effect of herstatin on IGF-I-stimulated cell proliferation. Herstatin expression blocked IGF-1-mediated survival of MCF7 cells. Growth of parental MCF7 breast carcinoma cells and MCF7 cells stably transfected with herstatin, (A) low hst-expressing clone, and (B) high hst-expressing clone, was determined by the MTS assay as described under Example 1 herein. Cells were serum-starved for 24 hours and then treated with 5 nM IGF-1 or vehicle, and growth was assessed at the indicated days.

### DETAILED DESCRIPTION OF THE INVENTION

Herstatin is the only known alternative receptor product that functions as a ligand, and is the only mammalian secreted ligand that inhibits members (HER-2 and EGFR) of the EGF receptor family (see, e.g., for background: Dougall et al., Oncogene 9:2109-23, 1994; Hynes & Stern, Biochim Biophys Acta 1198:165-84, 1994; and Tzahar & Yarden, Biochim Biophys Acta 1377:M25-37, 1998).

Aspects of the present invention describe and support HER-3, ΔEGFR, HER-4, and the IGF-IR as four additional (in addition to the previously disclosed binding to EGFR and HER-2) novel targets of herstatin and/or of its intron 8-encoded receptor binding domain (herein referred to as “Int8 RBD” or “RBD int8” polypeptide).

Additional aspects describe and support applicant's determination that intron 8 of the HER-2 gene, which is retained in an alternative HER-2 transcript (that encodes herstatin, encodes a 79-amino acid receptor binding domain (RBD) polypeptide (RBD Int8 polypeptide) that specifically binds to EGFR, HER-2, HER-3, ΔEGFR, HER-4, and the IGF-IR (RBD Int8 target receptors) with high affinity (e.g., nM affinity), but not to a mutant form of HER-3 having a substitution of Glu for Gly in the ectodomain of HER-3 at residue number 560, nor to the FGFR-3.

In particular aspects, as disclosed herein, herstatin inhibits target receptor-mediated activation of the intracellular signaling pathways (e.g., PI3/Akt, IRS-2, etc., pathways) that are important in cell survival, and further inhibit target receptor-mediated survival of cancer cells. Therefore, herstatin and/or RBD Int8 polypeptides and herstatin-, and/or RBD Int8 polypeptide-based agents (e.g., conjugates with toxins, radionuclides, etc.) have utility as therapeutic agents for treatment of diseases or conditions (e.g., cancer) characterized by cellular expression, or over-expression of a target receptor (e.g., of EGFR, HER-2, HER-3, ΔEGFR, HER-4, and/or the IGF-IR).

According to additional aspects, while the intron 8-encoded domain was demonstrated herein to be critical for receptor binding, it did not affect target receptor activity indicating that the N-terminal subdomains I and II of herstatin are likely required for receptor inhibition.

Furthermore, as disclosed herein, while the intron 8-encoded RBD appears to be critical for the receptor binding activity of herstatin, it is not conserved between humans and rats, despite a high degree of sequence identify between the HER-2 receptor and its rat ortholog, neu. Consistent with this result, there are distinct regions in the ectodomains of these two receptors that have very little identity (Stein and Staros, 2000).

According to particular aspects, therefore, the HER-3, HER-4 and ΔEGF receptors are specific targets of herstatin and/or the RBD Int8 polypeptide, likely based on specific binding of the RBD Int8-encoded domain. Moreover, and as in the case of the structurally related EGFR and HER-2 receptors, herstatin binds to and blocks the dimerization of the HER-3, HER-4 and ΔEGF receptors. As shown herein, for example, herstatin inhibits HER-4-mediated activation of the PI3/Akt pathway important in cells survival.

HER-3 is unique in the erbB family in that it is kinase-deficient, requiring an active receptor partner to signal. Additional aspects provide a mutant form of HER-3 that shows a lack of herstatin and/or RBD Int8 polypeptide binding. This mutant or variant form, therefore, has utility according to particular aspects of the present invention, for identification and/or screening of cells that are, at least to some extent, non-responsive, or at least less responsive to herstatin and/or RBD Int8 polypeptides, compared to cells expressing HER-3 forms that do bind herstatin and/or RBD Int8 polypeptides.

Surprisingly, according to particular aspects of the present invention, the IGF-1 receptor (IGF-1R) is also a specific target of herstatin and/or the RBD Int8 polypeptide, based on specific binding of the RBD Int8-encoded domain. The binding of herstatin and/or the RBD Int8 polypeptide to the IGF-IR with high affinity (e.g., nM affinity) was entirely unexpected, because receptor ligands do not typically cross-react with receptors from different families. Consistent with this result, however, the IGF-IR appears to have regions of ectodomain sequence homology with the EGFR, and it is known that “crosstalk” occurs between the families, most notably, ‘transactivation’ of the EGFR by IGF-1 (Ahmed T, Farnie N, et al., 2004; and references therein). Therefore, herstatin and/or RBD Int8 polypeptides and herstatin-, and/or RBD Int8 polypeptide-based agents (e.g., conjugates with toxins, radionuclides, etc.) have utility as therapeutic agents for treatment of diseases or conditions (e.g., cancer) characterized by cellular expression, or over-expression of the IGF-IR.

In particular determinations, the binding affinity of herstatin, but not of the RBD Int8 polypeptide, was found to be somewhat weaker for IGF-IR than for HER-2 or the EGFR, indicating less stabilizing interaction between the N-terminus of herstatin and the IGF-1 receptor ectodomain relative to the corresponding EGFR ectodomain regions (the IGF-IR does not have a homologous dimerization loop (Garrett et al., Cell 110:763-73, 2002).

According to additional aspects of the present invention, herstatin, the RBD Int8 polypeptide and herstatin- and/or RBD Int8 polypeptide-based agents can be used to target EGFR, HER-2, HER-3, DEGFR, HER-4 and IGF-IR, and/or modulate signaling mediated by these target receptors.

### DEFINITIONS

“Herstatin” refers to the polypeptides of SEQ ID NO:2, and additionally includes functional (e.g., target receptor-binding) variants (including conservative amino acid sequence variants as described herein), fragments, muteins, derivatives and fusion proteins thereof.

“RBD Int8 polypeptide” refers to the polypeptides of SEQ ID NO:1, and additionally includes functional (e.g., target receptor-binding) variants (including conservative amino acid sequence variants as described herein), fragments, muteins, derivatives and fusion proteins thereof

“Mutant RBD Int8 polypeptide” or “mutant Int8 RBD polypeptide” refers to the intron 8-encoded receptor binding domain variants (with an Arg to Ile mutation at residue 31 thereof) of SEQ ID NO:3), and additionally includes functional (e.g., target receptor non-binding) variants (including conservative amino acid sequence variants as described herein), fragments, muteins, derivatives and fusion proteins thereof. Representative, corresponding herstatin variants (Arg to Ile mutation at residue 371) are given as SEQ ID NO:4.

Functional herstatin, functional herstatin variants, functional Int8 RBD polypeptides, and functional Int8 RBD polypeptide variants are those proteins that display one or more of the biological activities of herstatin, including but not limited to target receptor binding, inhibition of receptor dimerization, modulation of receptor-mediated signal transduction, modulation of receptor activation, receptor down-regulation, etc. Particular aspects provide Functional herstatin, functional herstatin variants, functional Int8 RBD polypeptides, and functional Int8 RBD polypeptide variants having various binding affinities, including but not limited to those having a K_{D} of at least 20 nM, at least 40 nM, at least 60 nM, at least 80 nM, at least 100 nM, at least 120 nM, at least 140 nM, at least 160 nM, or at least 180 nM.

“EGFR,” “HER-1” or “erbB-1” refer to the art-recognized human epidermal growth factor receptor, erbB-1 (cDNA: NM_{—}005228, SEQ ID NO:5; protein: NP_{—}005219, SEQ ID NO:6), and including herstatin-, and/or Int8 RBD polypeptide-binding variants thereof.

“ΔEGFR” refers to the art-recognized receptor, ΔEGFR (cDNA: SEQ ID NO:7; protein: SEQ ID NO:8) (see Ekstrand et al., PNAS 89:4309-4313, 1992; and Nishikawa et al., PNAS 91:7727-7731, 1994) (comprising a deletion in the ECD; cDNA positions 275 through 1075, corresponding to exons 2-7 of the EGFR gene), and including herstatin-, and/or Int8 RBD polypeptide-binding variants thereof.

“HER-2” or “erbB-2” refers to the art-recognized human receptor, erbB-2 (cDNA: NM_{—}004448, SEQ ID NO:9; protein: NP_{—}004439, SEQ ID NO:10), and including herstatin-, and/or Int8 RBD polypeptide-binding variants thereof.

“HER-3” or “erbB-3” refers to the art-recognized human receptor, erbB-3 (cDNA: NM_{—}001982, SEQ ID NO:11; protein: NP_{—}001973, SEQ ID NO:12), and including herstatin-, and/or Int8 RBD polypeptide-binding variants thereof.

The phrase “mutant form of HER-3” refers to a HER-3 protein having a substitution of Glu for Gly in the ectodomain of HER-3 corresponding to a single point mutation at nucleotide position 1877 (“a” instead of “g” at this position), resulting in substitution of Glu instead of Gly at residue position 560) (cDNA: SEQ ID NO:13; protein: SEQ ID NO:14).

“HER-4” or “erbB-4” refers to the art-recognized human receptor, erbB-4 (cDNA: NM_{—}005235, SEQ ID NO:15; protein: NP_{—}005226, SEQ ID NO:16), and including herstatin-, and/or Int8 RBD polypeptide-binding variants thereof.

“IGF-1R” refers to the art recognized insulin-like growth factor 1 receptor (cDNA: NM_{—}000875, SEQ ID NO:17; protein: NP_{—}000866, SEQ ID NO:18), and including herstatin-, and/or Int8 RBD polypeptide-binding variants thereof.

As used herein, a pharmaceutical effect refers to an effect observed upon administration of an agent intended for treatment of a disease or disorder or for amelioration of the symptoms thereof.

As used herein, treatment means any manner in which the symptoms of a condition, disorder or disease or other indication, are ameliorated or otherwise beneficially altered.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to animals, including mammals, such as human beings. As used herein, a patient refers to a human subject.

As used herein, the phrase “associated with” refers to certain biological aspects such as expression of a receptor or signaling by a receptor that occurs in the context of a disease or condition. Such biological aspect may or may not be causative or integral to the disease or condition but merely an aspect of the disease or condition.

As used herein, a biological activity refers to a function of a polypeptide including but not limited to complexation, dimerization, multimerization, receptor-associated kinase activity, receptor-associated protease activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, membrane binding, and oncogenesis. A biological activity can be assessed by assays described herein and by any suitable assays known to those of skill in the art, including, but not limited to in vitro assays, including cell-based assays, in vivo assays, including assays in animal models for particular diseases.

TABLE 1
Summary of SEQ ID NOS and accession numbers:

| MOLECULE | cDNA | PROTEIN |
|---|---|---|
| RBD Int8 polypeptide(s)) | | SEQ ID NO: 1 |
| Herstatin (s) | | SEQ ID NO: 2 |
| Mutant Int8 RBD | | SEQ ID NO: 3 |
| polypeptide(s) | | |
| Mutant Herstatin (s) | | SEQ ED NO: 4 |
| EGFR (HER-1 or erbB-1) | SEQ ID NO: 5 (NM_005228) | SEQ ID NO: 6 (NP_005219) |
| ΔEGFR | SEQ ID NO: 7 | SEQ ID NO: 8 |
| HER-2 (erbB-2) | SEQ ID NO: 9 (NM_004448) | SEQ ID NO: 10 (NP_004439) |
| HER-3 (erbB-3) | SEQ ID NO: 11 (NM_001982) | SEQ ID NO: 12 (NP_001973) |
| Mutant form of HER-3 | SEQ ID NO: 13 | SEQ ID NO: 14 |
| HER-4 (erbB-4) | SEQ ID NO: 15 (NM_005235) | SEQ ID NO: 16 (NP_005226) |
| IGF-1R | SEQ ID NO: 17 (NM_000875) | SEQ ID NO: 18 (NP_000866) |

Herstatin and/or RBD Int8 Polypeptides and Therapeutic Agents

In preferred aspects, the present invention provides for the use of herstatin (SEQ ID NO:2), and variants and polypeptides thereof that bind to a target receptor (e.g., EGFR, HER-2, HER-3, DEGFR, HER-4 and IGF-IR). Also provided are uses of RBD Int8 polypeptides (SEQ ID NO:2), and receptor-binding variants and polypeptides thereof that bind to a target receptor (e.g., EGFR, HER-2, HER-3, DEGFR, HER-4 and IGF-IR).

Preferably, the herstatin, or variant thereof comprises an amino acid sequence of SEQ ID NO:2 (or of SEQ ID NO:2 having from 1, to about 3, to about 5, to about 10, or to about 20 conservative amino acid substitutions), or a fragment of a sequence of SEQ ID NO:2 (or of SEQ ID NO:2 having from 1, to about 3, to about 5, to about 10, or to about 20 conservative amino acid substitutions) of about 80 to 419 contiguous residues in length, wherein the C-terminal 79 contiguous amino acids are present, and wherein the polypeptide binds to the extracellular domain (ECD) of a target receptor (e.g., EGFR, HER-2, HER-3, DEGFR, HER-4 and IGF-IR) with an affinity binding constant of at least 10⁷ M⁻¹, at least 5×10⁷ M⁻¹, or at least 10⁸ M⁻¹. Preferably, the herstatin, or variant thereof, is from about 350 to 419 contiguous residues in length. Preferably, the herstatin, or variant thereof, binds to the extracellular domain (ECD) of a target receptor (e.g., EGFR, HER-2, HER-3, DEGFR, HER-4 and IGF-IR) with an affinity binding constant of at least 10⁷ M⁻¹, at least 5×10⁷ M⁻¹, or at least 10⁸ M⁻¹. Preferably, herstatin, or variant thereof, comprises a sequence of SEQ ID NO:2, or a conservative amino acid substitution variant thereof.

Preferably, the RBD Int8 polypeptides, and variants thereof, comprise an amino acid sequence of SEQ ID NO:1 (or of SEQ ID NO:1 having from 1, to about 3, to about 5, to about 10, or to about 20 conservative amino acid substitutions), or a fragment of a sequence of SEQ ID NO:1 (or of SEQ ID NO:1 having from 1, to about 3, to about 5, to about 10, or to about 20 conservative amino acid substitutions) of about 50 to 79 contiguous residues in length, wherein the polypeptide binds to the extracellular domain (ECD) of a target receptor (e.g., EGFR, HER-2, HER-3, DEGFR, HER-4 and IGF-IR) with an affinity binding constant of about 10⁷ M⁻¹, about 5×10⁷ M⁻¹, about 10⁸ M^{-1,} or greater (or at least 10⁷ M⁻¹, at least 5×10⁷ M⁻¹, or at least 10⁸ M⁻¹). Preferably, the RBD Int8 polypeptide, or variant thereof is from about 69 to 79 contiguous residues in length with a target receptor (e.g., EGFR, HER-2, HER-3, DEGFR, HER-4 and IGF-IR) affinity binding constant of about 10⁷ M⁻¹, about 5×10⁷ M⁻¹, about 10⁸ M⁻¹, or greater (or at least 10⁷ M⁻¹, at least 5×10⁷ M⁻¹, or at least 10⁸ M⁻¹). Preferably, the RBD Int8 polypeptide, or variant thereof comprises a sequence of SEQ ID NO:1, or a conservative amino acid substitution variant thereof.

### Specific Exemplary Embodiments

Methods of Treatment using a Herstatin, or a Variant Thereof

A preferred embodiment of the present invention provides a method for treating a condition characterized by altered cellular receptor expression or receptor-mediated signaling, comprising administering to a subject in need thereof, a therapeutically effective amount of a herstatin, or of a variant thereof, that binds to the extracellular domain of at least one target receptor of a target cell of the subject, wherein the at least one target receptor is selected from the group consisting of: ΔEGFR; HER-3 (erbB-3); HER-4 (erbB-4) and IGF-1.

In particular embodiments, the condition is a cellular proliferative condition or disorder, and preferably, the cellular proliferative condition or disorder is cancer.

In other embodiments, the target cell further expresses EGFR (HER-1, erbB-1), HER-2 (erbB-2) or both.

In particular embodiments, the herstatin, or variant thereof, comprises a polypeptide selected from the group consisting of SEQ ID NO:2, or a fragment of SEQ ID NO:2 of about 80 to 419 contiguous residues in length. Preferably, the herstatin, or variant thereof comprises the C-terminal 79 contiguous amino acids of SEQ ID NO:2, and binds to the extracellular domain of the at least one target receptor with an affinity binding constant of at least 10⁷ M⁻¹.

Further embodiment provide for application of the methods in instances where the cancer is refractory, at least to some extent, to treatment by at least one other therapeutic agent that is specific for a receptor selected from the group consisting of: EGFR (HER-1, erbB-1); ΔEGFR; HER-2 (erbB-2); HER-3 (erbB-3); HER-4 (erbB-4) and IGF-1, and wherein the at least one other therapeutic agent is different than herstatin, herstatin variants, int8 RDB polypeptides, and int8 RDB polypeptide variants.

Additional embodiments further comprise administering a therapeutically effective amount of a receptor-specific antibody that binds to the extracellular domain of a cellular receptor of the target cell. Preferably, the receptor-specific antibody binds to a cellular receptor selected from the group consisting of: EGFR (HER-1, erbB-1); ΔEGFR; HER-2 (erbB-2); HER-3 (erbB-3); HER-4 (erbB-4) and IGF-1. In particular embodiments, the receptor-specific antibody is the HER-2-specific antibody rhuMAb4D5 (HERCEPTIN™). In alternate embodiments, the receptor-specific antibody binds to a cellular receptor of the target cell that is different from the at least one cellular receptor bound by the herstatin, or the variant thereof. Preferably, the at least one other agent comprises a receptor-specific antibody, or a small-molecule receptor tyrosine kinase inhibitor.

Yet further embodiments comprise administration of a therapeutically effective amount of a chemotherapeutic agent. In particular embodiments, the chemotherapeutic agent is an anti-neoplastic agent selected from the group consisting of: cyclophosphamide, triethylenephosphoramide, triethylenethiophosphoramide, flutamide, altretamine, triethylenemelamine, trimethylolmelamine, meturedepa, uredepa, aminoglutethimide, L-asparaginase, BCNU, benzodepa, bleomycin, busulfan, camptothecin, capecitabine, carboquone, chlorambucil, cytarabine, dactinomycin, daunomycin, daunorubicin, docetaxol, doxorubicin, epirubicin, estramustine, dacarbazine, etoposide, fluorouracil, gemcitabine, hydroxyurea, ifosfamide, improsulfan, mercaptopurine, methotrexate, mitomycin, mitotane, mitoxantrone, novembrichin, paclitaxel, piposulfan, plicamycin, prednimustine, procarbazine, tamoxifen, temozolomide, teniposide, thioguanine, thiotepa, UFT, uracil mustard, vinblastine, vincristine, vinorelbine and vindesine.

In preferred embodiments, the herstatin, or variant thereof, comprises SEQ ID NO:23, which corresponds to the most common herstatin sequence (wild-type).

Methods of Treatment using an Int8 RBD Polypeptide, or a Variant Thereof

Alternate preferred embodiments provide a method for treating a condition characterized by altered cellular receptor expression or receptor-mediated signaling, comprising administering to a subject in need thereof, a therapeutically effective amount of an Int8 RBD polypeptide, or a variant thereof, that binds to the extracellular domain of at least one target receptor of a target cell of the subject, wherein the at least one target receptor is selected from the group consisting of: ΔEGFR; HER-3 (erbB-3); HER-4 (erbB-4) and IGF-1.

In particular embodiments, the condition is a cellular proliferative condition or disorder, and preferably the cellular proliferative condition or disorder is cancer.

In additional embodiments, the target cell further expresses EGFR (HER-1, erbB-1), HER-2 (erbB-2) or both.

In particular embodiments, the Int8 RBD polypeptide, or a variant thereof, comprises a polypeptide selected from the group consisting of SEQ ID NO:1, or a fragment of SEQ ID NO:1 of about 50 to 79 contiguous residues in length. Preferably, the Int8 RBD polypeptide, or a variant thereof binds to the extracellular domain of the at least one target receptor with an affinity binding constant of at least 10⁷ M⁻¹.

Further embodiments provide for application of the methods where the cancer is refractory, at least to some extent, to treatment by at least one other therapeutic agent that is specific for a receptor selected from the group consisting of: EGFR (HER-1, erbB-1); ΔEGFR; HER-2 (erbB-2); HER-3 (erbB-3); HER-4 (erbB-4) and IGF-1, and wherein the at least one other therapeutic agent is different than herstatin, herstatin variants, int8 RDB polypeptides, and int8 RDB polypeptide variants. Preferably, the at least one other agent comprises a receptor-specific antibody, or a small-molecule receptor tyrosine kinase inhibitor.

Additional embodiments further comprise administering a therapeutically effective amount of a receptor-specific antibody that binds to the extracellular domain of a cellular receptor of the target cell. In particular embodiments, the receptor-specific antibody binds to a cellular receptor selected from the group consisting of: EGFR (HER-1, erbB-1); ΔEGFR; HER-2 (erbB-2); HER-3 (erbB-3); HER-4 (erbB-4) and IGF-1. In a particular embodiment, the receptor-specific antibody is the HER-2-specific antibody rhuMAb4D5 (HERCEPTIN™). In alternate embodiments, the receptor-specific antibody binds to a cellular receptor of the target cell that is different from the at least one cellular receptor bound by the Int8 RBD polypeptide, or the variant thereof.

Yet additional embodiments further comprise administration of a therapeutically effective amount of a chemotherapeutic agent, and in particular embodiments, the chemotherapeutic agent is an anti-neoplastic agent selected from the group consisting of: cyclophosphamide, triethylenephosphoramide, triethylenethiophosphoramide, flutamide, altretamine, triethylenemelamine, trimethylolmelamine, meturedepa, uredepa, aminoglutethimide, L-asparaginase, BCNU, benzodepa, bleomycin, busulfan, camptothecin, capecitabine, carboquone, chlorambucil, cytarabine, dactinomycin, daunomycin, daunorubicin, docetaxol, doxorubicin, epirubicin, estramustine, dacarbazine, etoposide, fluorouracil, gemcitabine, hydroxyurea, ifosfamide, improsulfan, mercaptopurine, methotrexate, mitomycin, mitotane, mitoxantrone, novembrichin, paclitaxel, piposulfan, plicamycin, prednimustine, procarbazine, tamoxifen, temozolomide, teniposide, thioguanine, thiotepa, UFT, uracil mustard, vinblastine, vincristine, vinorelbine and vindesine.

In preferred embodiments, the Int8 RBD polypeptide, or variant thereof, comprises SEQ ID NO:24, which corresponds to the most common Int8 RBD polypeptide sequence (wild-type).

Methods of Cellular Targeting

Yet further embodiments provide a method for targeting a therapeutic agent to target cells, comprising attaching the therapeutic agent to herstatin, or to a variant thereof, that binds to the extracellular domain of at least one target receptor of a target cell, wherein the at least one target receptor is selected from the group consisting of: ΔEGFR; HER-3 (erbB-3); HER-4 (erbB-4) and IGF-1.

In particular embodiments, the target cell is a cancer cell.

In other embodiments the target cell optionally further expresses EGFR (HER-1, erbB-1), HER-2 (erbB-2) or both.

In particular embodiments, the herstatin, or variant thereof, comprises a polypeptide selected from the group consisting of SEQ ID NO:2, or a fragment of SEQ ID NO:2 of about 80 to 419 contiguous residues in length. Preferably, the herstatin, or variant thereof comprises the C-terminal 79 contiguous amino acids of SEQ ID NO:2, and binds to the extracellular domain of the at least one target receptor with an affinity binding constant of at least 10⁷ M⁻¹.

Alternate embodiments provide a method for targeting a therapeutic agent to target cells, comprising attaching the therapeutic agent to an Int8 RBD polypeptide, or to a variant thereof, that binds to the extracellular domain of at least one target receptor of a target cell, wherein the at least one target receptor is selected from the group consisting of: ΔEGFR; HER-3 (erbB-3); HER-4 (erbB-4) and IGF-1.

In particular embodiments, the target cell is a cancer cell.

In other embodiments, the target cell further expresses EGFR (HER-1, erbB-1), HER-2 (erbB-2) or both.

In particular embodiments, the Int8 RBD polypeptide, or a variant thereof, comprises a polypeptide selected from the group consisting of SEQ ID NO:1, or a fragment of SEQ ID NO:1 of about 50 to 79 contiguous residues in length. Preferably, the Int8 RBD polypeptide, or a variant thereof binds to the extracellular domain of the at least one target receptor with an affinity binding constant of at least 10⁷ M⁻¹.

Pharmaceutical Compositions

Yet additional embodiments provide pharmaceutical composition for treating a condition characterized by altered cellular receptor expression or receptor-mediated signaling, comprising, along with a pharmaceutically acceptable carrier or excipient, an agent selected from the group consisting of: (a) herstatin, or a variant thereof, that binds to the extracellular domain of at least one target receptor of a target cell, wherein the at least one target receptor is selected from the group consisting of: ΔEGFR; HER-3 (erbB-3); HER-4 (erbB-4) and IGF-1; (b) a Int8 RBD polypeptide, or a variant thereof, that binds to the extracellular domain of at least one target receptor of a target cell, wherein the at least one target receptor is selected from the group consisting of: ΔEGFR; HER-3 (erbB-3); HER-4 (erbB-4) and IGF-1; (c) a receptor-specific antibody that binds to the extracellular domain (ECD) of a cellular receptor of the target cell; and (d) combinations thereof, with the proviso that where the composition comprises the target cell receptor-specific antibody it also comprises at least one of the agents of (a) or (b).

Additional embodiments provide for a pharmaceutical composition for treating a condition characterized by altered cellular receptor expression or receptor-mediated signaling, comprising, along with a pharmaceutically acceptable carrier or excipient, a first agent selected from the group consisting of: herstatin, or a variant thereof; a Int8 RBD polypeptide, or a variant thereof; and combinations thereof, the composition further comprising a second agent selected from the group consisting of: a receptor-specific antibody that binds to the extracellular domain (ECD) of a cellular receptor of the target cell; a small molecule receptor tyrosine kinase inhibitor; and combinations thereof, with the proviso that the receptor-specific antibody is not a HER-1 or HER-2-specific antibody.

Preferably, the herstatin, or variant thereof, comprises SEQ ID NO:23. Preferably, the Int8 RBD polypeptide, or variant thereof, comprises SEQ ID NO:24.

In particular embodiments, the condition treated with the composition is a cellular proliferative condition or disorder, and preferably the cellular proliferative condition or disorder is cancer.

In additional embodiments, the target cell further expresses EGFR (HER-1, erbB-1), HER-2 (erbB-2) or both.

In particular embodiments, when agent (c) is present, the receptor-specific antibody binds to a cellular receptor of the target cell that is different from the at least one cellular receptor bound by the other agents (a) or (b).

In preferred embodiments agent (a) the herstatin, or variant thereof, comprises a polypeptide selected from the group consisting of SEQ ID NO:2, or a fragment of SEQ ID NO:2 of about 80 to 419 contiguous residues in length, and agent (b) the Int8 RBD polypeptide, or a variant thereof, comprises a polypeptide selected from the group consisting of SEQ ID NO:1, or a fragment of SEQ ID NO:1 of about 50 to 79 contiguous residues in length.

Further embodiments provide for a pharmaceutical composition for treating a condition characterized by altered cellular receptor expression or receptor-mediated signaling, comprising, along with a pharmaceutically acceptable carrier or excipient, a polynucleotide that encodes a herstatin, or a herstatin variant.

Yet further embodiments provide for a pharmaceutical composition for treating a condition characterized by altered cellular receptor expression or receptor-mediated signaling, comprising, along with a pharmaceutically acceptable carrier or excipient, a polynucleotide that encodes an int8 RBD polypeptide, or an int8 RBD polypeptide variant.

Mutant/Variant HER-3 Screening Assays

Particular embodiments provide for a method for identification of cells having HER-3 receptors that do not bind herstatin, int 8 RDB polypeptides, or variants thereof, comprising: obtaining a cellular sample; and determining, using one or more suitable assays, whether the cells express SEQ ID NO:14.

Additional embodiments provide for screening for cells that are, at least to some extent, non-responsive to herstatin, int 8 RDB polypeptides, or variants thereof, comprising obtaining a cellular sample; and determining, using one or more suitable assays, wherein the cells are determined to be at least to some extent, non-responsive to herstatin, int 8 RDB polypeptides, or variants thereof, express SEQ ID NO:14, wherein if the cells express SEQ ID NO:14.

Biologically Active Variants

Functional herstatin, functional herstatin variants, functional Int8 RBD polypeptides, and functional Int8 RBD polypeptide variants are those proteins that display one or more of the biological activities of herstatin, including but not limited to target receptor binding, inhibition of receptor dimerization, modulation of receptor-mediated signal transduction, modulation of receptor activation, receptor down-regulation, etc.

Variants of herstatin and/or RBD Int8 polypeptide have utility for aspects of the present invention. Variants can be naturally or non-naturally occurring. Naturally occurring variants (e.g., polymorphisms) are found in humans or other species and comprise amino acid sequences which are substantially identical to the amino acid sequence shown in SEQ ID NO:1 or 2. Species homologs of the protein can be obtained using subgenomic polynucleotides of the invention, as described below, to make suitable probes or primers for screening cDNA expression libraries from other species, such as mice, monkeys, yeast, or bacteria, identifying cDNAs which encode homologs of the protein, and expressing the cDNAs as is known in the art.

Non-naturally occurring variants which retain substantially the same biological activities as naturally occurring protein variants, specifically the target RBD activity and the modulation of target receptor signaling activity, are also included here. Preferably, naturally or non-naturally occurring variants have amino acid sequences which are at least 85%, 90%, or 95% identical to the amino acid sequence shown in SEQ ID NOS:1 or 2. More preferably, the molecules are at least 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. 2:482-489, 1981.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. .§§. 1.821-1.822, abbreviations for amino acid residues are shown in Table 2:

TABLE 2
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Praline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

It should be noted that all amino acid residue sequences represented herein by a formula have a left to right orientation in the conventional direction of amino-terminus to carboxyl-terminus. In addition, the phrase “amino acid residue” is defined to include the amino acids listed in the Table of Correspondence and modified and unusual amino acids, such as those referred to in 37 C.F.R. .§§ 1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

Guidance in determining which amino acid residues can be substituted, inserted, or deleted without abolishing biological or immunological activity can be found using computer programs well known in the art, such as DNASTAR software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant.

Variants of the herstatin and/or RBD Int8 polypeptide disclosed herein include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N— or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins (see, e.g., Mark et al., U.S. Pat. No. 4,959,314).

Preferably, amino acid changes in the herstatin and/or RBD Int8 polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of herstatin and/or RBD Int8 polypeptide protein or polypeptide variants are of the same type as a protein comprising the amino acid sequence encoded by the nucleotide sequence shown in SEQ ID NO:1 or 2, although the properties and functions of variants can differ in degree.

Herstatin and/or RBD Int8 polypeptide variants include glycosylated forms, aggregative conjugates with other molecules, and covalent conjugates with unrelated chemical moieties (e.g., pegylated molecules). Herstatin and/or RBD Int8 polypeptide variants also include allelic variants, species variants, and muteins. Truncations or deletions of regions which do not affect functional activity of the proteins are also variants. Covalent variants can be prepared by linking functionalities to groups which are found in the amino acid chain or at the N— or C-terminal residue, as is known in the art.

It will be recognized in the art that some amino acid sequences of the herstatin and/or RBD Int8 polypeptides of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors (Ostade et al., Nature 361:266-268, 1993). Thus, the herstatin and/or RBD Int8 polypeptides of the present invention may include one or more amino acid substitutions, deletions or additions, either from natural mutations or human manipulation.

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic (see, e.g., Pinckard et al., Clin. Exp. Immunol. 2:331-340 (1967); Robbins et al., Diabetes 36:838-845 (1987); and Cleland et al., Crit. Rev. Therapeutic Drug Carrier Systems 10:307-377 (1993)).

Amino acids in the herstatin and/or RBD Int8 polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given herstatin and/or RBD Int8 polypeptide will not be more than 50, 40, 30, 25, 20, 15, 10, 5 or 3.

In addition, pegylation of herstatin and/or RBD Int8 polypeptides and/or muteins is expected to provide such improved properties as increased half-life, solubility, and protease resistance. Pegylation is well known in the art.

Fusion Proteins

Fusion proteins comprising proteins or polypeptide fragments of herstatin and/or RBD Int8 polypeptide can also be constructed. Fusion proteins are useful for generating antibodies against amino acid sequences and for use in various targeting and assay systems. For example, fusion proteins can be used to identify proteins which interact with a herstatin and/or RBD Int8 polypeptide of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence can be used.

A fusion protein comprises two protein segments fused together by means of a peptide bond. Amino acid sequences for use in fusion proteins of the invention can be utilize the amino acid sequence shown in SEQ ID NO:1 or 2 or can be prepared from biologically active variants of SEQ ID NO:1 or 2, such as those described above. The first protein segment can include of a full-length herstatin and/or RBD Int8 polypeptide.

Other first protein segments can consist of about 50 to about 79 contiguous amino acids from SEQ ID NO:1, or, with respect to SEQ ID NO:2, from about 80 to 419 contiguous residues in length, wherein the C-terminal 79 contiguous amino acids of SEQ ID NO:2 are present, or from about 350 to 419 contiguous residues in length wherein the C-terminal 79 contiguous amino acids of SEQ ID NO:2 are present.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding region for the protein sequence of SEQ ID NO:1 or 2 in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Cell Targeting

According to particular aspects of the present invention, herstatin- and/or RBD Int8 polypeptide-based agents can be used to target EGFR (HER-1, erbB-1); HER-2 (erbB-2); HER-3 (erbB-3); HER-4 (erbB-4), ΔEGFR or IGF-1R on cells (e.g., cancer cells). Herstatin- and/or RBD Int8 polypeptide-based agents can be used to deliver a locally acting biological agent that will affect the targeted cell.

Each of the target receptors (e.g., EGFR (HER-1, erbB-1); HER-2 (erbB-2); HER-3 (erbB-3); HER-4 (erbB-4), ΔEGFR or IGF-1R) is expressed on the surface of cells and are accessible to exogenous molecules. Where any of these target receptors are present at higher levels on cancer cells as compared to non-cancer cells, they can be utilized as preferential targets for systemic herstatin- and/or RBD Int8 polypeptide-based agents-based therapies. The differential expression of these target receptors enables the specificity of herstatin- and/or RBD Int8 polypeptide-based agents-based therapy. Herstatin- and/or RBD Int8 polypeptide-based cytotoxic agents directed against the target receptor preferentially affect cancer cells over normal tissue. For example, an herstatin- or RBD Int8 polypeptide-radioisotope conjugate that binds a target receptor (e.g., EGFR (HER-1, erbB-1); HER-2 (erbB-2); HER-3 (erbB-3); HER-4 (erbB-4), ΔEGFR or IGF-1R) present predominantly on cancer cells would be expected to selectively affect those cells within a treated individual. Preferably, the target is accessible to the herstatin- and/or RBD Int8 polypeptide-based agent, and is found in substantially greater concentrations on the targeted cancer cells than non-cancer cells.

Therefore, the present invention includes™—and/or RBD Int8 polypeptide-based agents specific to one or more of the target receptors that will enable or facilitate treatment of cancer.

In particular aspects, herstatin- and/or RBD Int8 polypeptides are conjugated or coupled to toxins.

In alternate embodiments, herstatin- and/or RBD Int8 polypeptides are conjugated or coupled to radionuclides.

Additional embodiments provide for herstatin- and/or RBD Int8 polypeptide-coated liposomes which contain one or more biologically active compounds.

In particular aspects, binding of an herstatin- and/or RBD Int8 polypeptide-agent to a cell is sufficient to inhibit growth (e.g., cytostatic effects) or kill the target cell (cytotoxic effects). The mechanism of these activities may vary, but may involve herstatin- and/or RBD Int8 polypeptide-dependent cell-mediated cytotoxicity, activation of apoptosis, inhibition of ligand-receptor function, or provide a signal for complement fixation. In fact, herstatin- and/or RBD Int8 polypeptide-agents may exhibit one or several of such activities. In particular aspects, herstatin- and/or RBD Int8 polypeptide-agents are cytostatic, but not cytotoxic. Preferably, herstatin and/or RBD Int8 polypeptide-agents bind to target receptors (e.g., EGFR (HER-1, erbB-1); HER-2 (erbB-2); HER-3 (erbB-3); HER-4 (erbB-4), ΔEGFR or IGF-1R), and are either cytoxic or cytostatic.

In particular embodiments, herstatin- and/or RBD Int8 polypeptide-agents can are conjugated or coupled to a diverse array of compounds which include, but are not limited to proteins, toxins or cytotoxic agents, radionuclides, apoptotic factors), anti-angiogenic compounds or other biologically active compounds which will inhibit the growth of or kill the target cell or tissue. For example, cytotoxic or cytostatic agents include, but are not limited to, diphtheria toxin and Pseudomonas exotoxin, ricin, gelonin, doxorubicin and its derivatives, iodine-131, yttrium-90, indium-111, RNAse, calicheamicin, apoptotic agents, and antiangiogenic agents. According to aspects of the present invention, herstatin- and/or RBD Int8 polypeptides coupled to these compounds are used to adversely affect cells displaying one or more target receptors (e.g., EGFR (HER-1, erbB-1); HER-2 (erbB-2); HER-3 (erbB-3); HER-4 (erbB-4), ΔEGFR or IGF-1R).

Toxins can also be targeted to specific cells by incorporation of the toxin into herstatin- and/or RBD Int8 polypeptide-coated liposomes. The herstatin- and/or RBD Int8 polypeptide-based agent directs the liposome to the target cell where the bioactive compound is released. For example, cytotoxins in herstatin- and/or RBD Int8 polypeptide-coated liposomes are used to treat cancer. In alternate embodiments, these targeted liposomes are loaded with DNA encoding bioactive polypeptides (e.g., inducible nitric oxide synthase).

Prodrugs or enzymes can also be delivered to targeted cells by specific herstatin- and/or RBD Int8 polypeptide-agents. In this case the herstatin conjugate consists of an herstatin- and/or RBD Int8 polypeptide-based agent coupled to a drug that can be activated once the antibody binds the target cell. Examples of this strategy using antibodies have been reviewed (e.g., Denny 2001; and Xu and McLeod 2001).

Therefore, in particular embodiments, herstatin- and/or RBD Int8 polypeptide-prodrug/enzyme conjugates targeted to one or more target receptors (e.g., EGFR (HER-1, erbB-1); HER-2 (erbB-2); HER-3 (erbB-3); HER-4 (erbB-4), ΔEGFR or IGF-1R) have utility for the treatment of cancer.

The specificity and high affinity of the herstatin- and/or RBD Int8 polypeptide-based agents makes them ideal candidates for delivery of toxic agents to a specific subset of cellular targets. Preferably, one or more target receptors (e.g., EGFR (HER-1, erbB-1); HER-2 (erbB-2); HER-3 (erbB-3); HER-4 (erbB-4), ΔEGFR or IGF-1R) are present at higher levels on the target cells (e.g., cancer, tumor cells) than on non-cancer cells.

Pharmaceutical Compositions and Therapeutic Uses

Pharmaceutical compositions of the invention can comprise herstatin and/or RBD Int8 polypeptides, or herstatin- and/or RBD Int8 polypeptide-based agents of the claimed invention in a therapeutically effective amount. The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/kg to 50 mg/kg or 0.05 mg/kg to about 10 mg/kg of the herstatin and/or RBD Int8 polypeptide constructs in the individual to which it is administered. A non-limiting example of a pharmaceutical composition is a composition that either enhances or diminishes signaling mediated by the inventive target receptors (e.g., EGFR, HER-2, HER-3, DEGFR, HER-4 and IGF-IR). Where such signaling promotes a disease-related process, modulation of the signaling would be the goal of the therapy.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., New Jersey, 1991).

Delivery Methods. Once formulated, the compositions of the invention can be administered directly to the subject or delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, myocardial, intratumoral, peritumoral, or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, direct microinjection of the DNA into nuclei, and viral-mediated, such as adenovirus or alphavirus, all well known in the art.

In a preferred embodiment, disorders of proliferation, such as cancer, can be amenable to treatment by administration of a therapeutic agent based on the provided polynucleotide or corresponding polypeptide. The therapeutic agent can be administered in conjunction with one or more other agents including, but not limited to, receptor-specific antibodies and/or chemotherapeutic (e.g., anti-neoplastic agents). Administered “in conjunction” includes administration at the same time, or within 1 day, 12 hours, 6 hours, one hour, or less than one hour, as the other therapeutic agent(s). The compositions may be mixed for co-administration, or may be administered separately by the same or different routes.

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic compositions agents of the invention includes local or systemic administration, including injection, oral administration, particle gun or catheterized administration, and topical administration. The therapeutic polynucleotide composition can contain an expression construct comprising a promoter operably linked to a polynucleotide encoding, for example, SEQ ID NO:2, or encoding about 80 to 419 (or about 350 to 419) contiguous amino acids of SEQ ID NO:2. Various methods can be used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. X-ray imaging is used to assist in certain of the above delivery methods.

Herstatin and/or RBD Int8 polypeptide-mediated targeted delivery of therapeutic agents to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 mg to about 2 mg, about 5 mg to about 500 mg, and about 20 mg to about 100 mg of DNA can also be used during a gene therapy protocol. Factors such as method of action (e.g., for enhancing or inhibiting levels of the encoded gene product) and efficacy of transformation and expression are considerations which will affect the dosage required for ultimate efficacy of the subgenomic polynucleotides. Where greater expression is desired over a larger area of tissue, larger amounts of subgenomic polynucleotides or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5, 219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. 264:16985 (1989)); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. 14:2411 (1994), and in Woffendin, Proc. Natl. Acad. Sci. (1994) 91:11581-11585.

Further non-viral delivery suitable for use includes mechanical delivery systems such as the approach described in Woffendin et al., Proc. Natl. Acad. Sci. USA 91(24):11581 (1994). Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

Exemplary Conditions Treatable and Combination Therapies

The present invention, for the first time, not only discloses that herstatin and/or the intron 8-encoded domain thereof (referred to herein as “int8 RBD”polypeptides), and variants thereof, not only bind with high affinity (e.g., at nM concentrations) to: all four of the ErbB receptors EGFR (HER-1, erbB-1), HER-2 (erbB-2), HER-3 (erbB-3), and HER-4 (erbB-4), and to ΔEGFR and the IGF-1 receptor, but also discloses that such target receptor binding has novel and substantial utility to modulate intracellular signaling mediated by these receptors.

Therefore, the present invention encompasses a broad range of utilities, including therapeutic utilities. For example, particular embodiments provide novel methods and compositions for the treatment of cancer and other conditions and disorders characterized by target receptor expression or over-expression, and/or target receptor-mediated signaling or aberrant signaling.

Specific embodiments provide a method for treating cancer, comprising administering a therapeutically effective amount of herstatin, or of a variant thereof, that binds to the extracellular domain of a target receptor selected from the group consisting of: ΔEGFR; HER-3 (erbB-3); HER-4 (erbB-4), IGF-1R and combinations thereof, wherein the cancer cells express at least one of the target receptors. Alternatively, a therapeutically effective amount of a Int8 RBD polypeptide, or of a variant thereof, that binds to the extracellular domain of a target receptor selected from the group consisting of: Δ EGFR; HER-3 (erbB-3); HER-4 (erbB-4), IGF-1R and combinations thereof, is administered. The methods also encompass treatments where the cancer cells further express EGFR (HER-1, erbB-1), HER-2 (erbB-2) or both.

Combination therapies are also encompassed by aspects of the present invention. For example, the inventive methods may further comprise administration of a therapeutically effective amount of: a receptor-specific antibody that binds to the extracellular domain of a target receptor selected from the group consisting of: EGFR (HER-1, erbB-1); Δ EGFR; HER-2 (erbB-2); HER-3 (erbB-3); HER-4 (erbB-4), and IGF-1R. Alternatively, the inventive methods may further comprise administration of chemotherapeutic agents, such as antineoplastic agents. Examples of anti-neoplastic agents are cyclophosphamide, triethylenephosphoramide, triethylenethiophosphoramide, flutamide, altretamine, triethylenemelamine, trimethylolmelamine, meturedepa, uredepa, aminoglutethimide, L-asparaginase, BCNU, benzodepa, bleomycin, busulfan, camptothecin, capecitabine, carboquone, chlorambucil, cytarabine, dactinomycin, daunomycin, daunorubicin, docetaxol, doxorubicin, epirubicin, estramustine, dacarbazine, etoposide, fluorouracil, gemcitabine, hydroxyurea, ifosfamide, improsulfan, mercaptopurine, methotrexate, mitomycin, mitotane, mitoxantrone, novembrichin, paclitaxel, piposulfan, plicamycin, prednimustine, procarbazine, tamoxifen, temozolomide, teniposide, thioguanine, thiotepa, UFT, uracil mustard, vinblastine, vincristine, vinorelbine and vindesine.

Treatment of refractory cancer. By virtue of their activation of the PI3K and MAPK cascades and potentially other signal transduction pathways, both the EGF and IGF receptor families are major regulators of cell growth and survival, and dysregulation of either receptor family can lead to uncontrolled growth and tumorgenesis. Moreover, ‘cross-talk’ is believed to occur between these receptor families, and various studies support the concept that redundant signaling through IGF-IR maintains activation of critical pathways for survival in the presence of EGFR family inhibitors. Such cross-talk and redundant signaling has been shown to be involved in cancers that are, or that become refractory to treatment by, for example, a particular receptor-specific agent (e.g., antibody reagent, or small molecule receptor tyrosine kinase inhibitor) or class of agents; that is, such cancers do not respond, respond only weakly, or progressively become less responsive to particular agents, by virtue of intracellular signaling mediated by a receptor other than the one being targeted by the particular agent. These findings all point to the need to for a multi-functional inhibitor that simultaneously targets both the EGF and IGF-IR families. Aspects of the present invention have met this need.

Accordingly, further embodiments provide for application of the methods where the cancer is refractory, at least to some extent, to treatment by at least one other therapeutic agent that is specific for a receptor selected from the group consisting of: EGFR (HER-1, erbB-1); ΔEGFR; HER-2 (erbB-2); HER-3 (erbB-3); HER-4 (erbB-4) and IGF-1, and wherein the at least one other therapeutic agent is different than herstatin, herstatin variants, int8 RDB polypeptides, and int8 RDB polypeptide variants. Preferably, the at least one other agent comprises a receptor-specific antibody, or a small-molecule receptor tyrosine kinase inhibitor.

According to the present invention therefore, herstatin or Int8 RBD polypeptides, and variants thereof can be used in therapeutic methods and pharmaceutical compositions to treat a variety of conditions having an aspect related to, or associated with altered target receptor expression, altered target receptor expression, target receptor-mediated signaling, or altered target receptor-mediated signaling at a cellular level. Such methods comprising administering to a subject having such a condition, a therapeutically effective amount of a herstatin or Int8 RBD polypeptide, or a variant thereof, that binds to the extracellular domain of at least one cellular target receptor.

The present invention will now be illustrated by reference to the following examples which set forth particularly advantageous embodiments. However, it should be noted that these embodiments are illustrative and are not to be construed as restricting the invention in any way.

### EXAMPLE 1

### Materials and Methods

Cell Lines, Transfections, Expression Vectors, Western Blots and Antibodies

Cell lines. The 3T3/HER-2 cells were previously described (Lin et al., Mol. Cell. Endocrinol., 69:111-9, 1990). The 3T3/IGF-IR cells were from Dr. Charles Roberts, OHSU, Portland, Oreg. MCF7 breast carcinoma cells were obtained from the American Type Culture Collection and maintained at 37° C./5% CO₂ in Dulbecco's modified Eagle's medium (DMEM) containing 10% fetal bovine serum (FBS) and gentamicin (0.25 μg/ml). Media and supplements were purchased from Gibco BRL-Life Technologies (Grand Island, N.Y.). Hst-expressing MCF7 clones (previously characterized in Jhabvala-Romero et al., Oncogene 22:8178-86, 2003), were maintained under the same conditions as parental MCF7 cells in media supplemented with 0.5 mg/ml G418 sulfate.

Transfections. For transient transfections, 2 μg of empty vector or 2 μg EGFR, HER-2, HER-3, HER-4, ΔEGFR, or FGFR-3-myc expression vectors were added with Lipofectamine™ (GIBCO-BRL) to Cos-7 cells in 6 cm plates.

Expression vectors. The HER-2 and EGFR expression plasmids were previously described (Azios et al., Oncogene 20:5199-209, 2001), ΔEGFR was a gift from Dr. Webster Cavenee (Ludwig Institute for Cancer Research, UCSD, La Jolla, Calif.), the FGFR-3-myc construct was from Dr. William Horton (Shriners Research Hospital, Portland, Oreg.), and the HER-4 expression plasmid was a gift of Dr. Nancy Hynes (Friedrich Miescher-Institute for Biomedial Research, Basel, Switzerland).

Antibodies. Antibodies against the β-subunit of IGF-IR were from Dr. Charles Roberts (Oregon Health & Science University). All primary antibodies were used at a 1:1000 dilution and incubated with Western blots overnight at 4° C., unless otherwise indicated. Polyclonal antibodies (IGF-IR and IRS-1) and monoclonal antibody PY20 were obtained from Santa Cruz Biotechnology (Santa Cruz, Calif.). Monoclonal ERK 1/2 and polyclonal pERK 1/2 and Akt/PKB antibodies were purchased from Cell Signaling Technologies (Boston, Mass.). Monoclonal herstatin and polyclonal IRS-2 antibodies were obtained from Upstate Biotechnology (Lake Placid, N.Y.). Polyclonal pAkt/PKB and pIGF-IR antibodies were purchased from Biosource International (Hopkinton, Mass.) and polyclonal anti-Shc antibody was obtained from Transduction Labs (Lexington, Ky.).

Western blot analysis. To analyze receptors by Western blot analysis, proteins were resolved by SDS-PAGE and electro-transferred onto nitrocellulose membranes (BioRad, Hercules, Calif.). Blots were blocked in 5% milk and incubated with primary antibody overnight at 4° C. The antibodies included anti-HER-2 (Christianson et al., Cancer Res. 58:5123-9, 1998) and anti-EGFR, anti-HER-3, anti-HER-4, which were all rabbit polyclonal antibodies against the receptor C-terminal domains (Santa Cruz Biotechnology). After washing, the blots were incubated with secondary antibody conjugated to HRP for 30 min (BioRad, Hercules, Calif.). The membranes were developed with SuperSignal™ West Dura (Pierce, Rockford, Ill.) and exposed to x-ray film. In particular studies, cells were grown to ˜80% confluency, serum-starved overnight in DMEM, and treated with 14 nM EGF, 5 nM IGF-I, or 20 nM IGF-II (in some experiments) for the times indicated. For Western immunoblots, cells were washed twice with ice-cold 1×PBS and lysed in 1×SDS sample buffer (Maniatis) without DTT or dye and boiled for 5 min. After clarification by centrifugation at 13,000 rpm for 5 min., protein concentration was determined using a detergent-compatible protein assay kit (Bio-Rad; Hercules, Calif.). DTT was then added to 100 mM and bromophenol blue to 0.1% (w/v) and samples were boiled again for 5 min. 20 mg protein was run on a 10% SDS-PAGE and blotted onto nitrocellulose (Amersham Pharmacia Biotech; Piscataway, N.J.). Blots were probed with a phospho-specific antibody, stripped in 5× stripping buffer (Maniatis) and reprobed with the respective pan antibody. For immunoprecipitation, cells were washed twice with ice-cold 1×PBS, lysed in NP-40 lysis buffer [1% NP-40, 150 mM NaCl, 10% glycerol, 20 mM Tris-HCl (pH 8.0), 1 mM EDTA (pH 8.0), 0.2% SDS, complete protease inhibitors (Roche Diagnostics; Indianapolis, Ind.), 1 mM NaVO₄, and 1 mg/ml pepstatin] and kept on ice for 30 min, inverting the tubes every 2 minutes. Lysates were then centrifuged at 13,000 rpm for 15 minutes and the supernatant transferred to a new tube. Protein concentration was determined as above. For IGF-IR, 1 mg of whole-cell lysate protein was immunoprecipitated with 16 mg of anti-IGF-IR antibody and incubated overnight at 4° C. while rocking. For IRS-1 and IRS-2, 500 mg of whole-cell lysate protein was incubated overnight with 10 mg antibody. 100 ml of protein A-agarose bead slurry (Amersham Pharmacia Biotech) was added for 2 hours rocking at 4° C. Three washes were performed and the pellet was boiled in 2×SDS sample buffer (Maniatis). The beads were spun down and the supernatant loaded onto a 10% (IGF-IR) or 7% (IRS-1/2) SDS-PAGE and blotted as above. Blots were probed with PY20, stripped as above, and reprobed with their respective antibodies. Binding of primary antibodies was detected by enhanced chemiluminescence (Amersham), and film exposures were quantified using a scanning densitometer (Bio-Rad).

Sequencing of Human, Monkey and Rat Intron 8 Regions:

Human. Human genomic DNA was obtained from blood samples (supplied by Dr. David Henner, OHSU) from individuals 18 years or more, after giving informed consent, with approval by the Institutional Review Board of OHSU. The samples, assigned random four-digit numbers, could not be traced to patient identity. The polymerase chain reaction (PCR) was employed to amplify intron 8 using primers: 3′ AACACAGCGGTGTGAGAAGTGC (exon 8) (SEQ ID NO:19) and 5′ GTATCGGTAGTTCATTTCCTTTGGTTGC (intron 9) (SEQ ID NO:20). The reactions were cycled (95° C. for 2 minutes, 95° C. for 30 seconds, 69° C. for 30 seconds, 72° C. for 30 seconds) for 30 cycles. PCR products were purified and subjected to cycle-sequencing. Electropherograms were individually reviewed to detect polymorphic alleles. Samples found to contain a polymorphism were sequenced at least once more to confirm the mutation.

Monkey. Rhesus monkey DNA, provided by Dr. Scott Wong (ORPC, Portland, Oreg.) was amplified and sequenced using the above primers.

Rat. Intron 8 in rat genomic DNA (provided by Dr. John Adelman, Vollum Institute, Portland, Oreg.) was amplified by PCR using rat specific primers: 5′-CTA CCT GTC TAC GGA AGT GG-3′ (SEQ ID NO:21) and 5′-TTC CGG GCA GAA ATG CCA GG-3′ (SEQ ID NO:22). The cycling parameters were: 94° C., 30″; 62° C., 30″; 72° C., 60″, for 25 cycles.

Expression and Purification of Intron 8-Encoded Peptide (Int8) and Herstatin:

Receptor binding domain (RBD). Intron 8 cDNA was cloned into the pET 30 bacterial expression vector (Novagen, Madison, Wis.), expressed in bacteria (BL-21), and purified by nickel affinity chromatography as described (Doherty et al., Supra).

Herstatin. For purification of insect herstatin, S2 insect cells, stably transfected with 6×His tagged-herstatin in the pMT/BiP expression plasmid (Invitrogen, Carlsbad, Calif.), were induced with 100 μM cupric sulfate for about 16 hrs. Herstatin was purified to about 90% purity by Ni—NTA (Qiagen, Valencia, Calif.) affinity chromatography as previously described (Jhabvala-Romero et al. Supra.).

Cell Binding Studies:

ELISA. Monolayer cultures of ˜2×10⁶ cells were plated in 6-well tissue culture plates, and were incubated with purified herstatin or int8 peptide for 2 hours at 4° C. in serum-free DMEM. Cells were washed with Phosphate Buffered Saline (PBS) and extracted in 50 mM Tris-HCl, pH 7.0, 1.0% NP-40. Int8 peptide or herstatin bound to cells were quantified using a sandwich herstatin ELISA per manufacturer's instructions (Upstate Biotechnology, Lake Placid, N.Y.).

The dissociation constant (K_{D}) and maximal binding (Bₘₐₓ) of herstatin or the int8 peptide were determined by nonlinear regression analysis of the plot of pmol of bound versus nM of herstatin or int8 peptide added. Statistical comparisons between different binding curves were performed by extra sums-of-squares F-test nonlinear regression coefficients. All tests were performed (α=0.05) using GraphPad Prism 4™ software (GraphPad™ Software, 1994-2003).

Pull-Down Assays with Int8 Peptide Immobilized on Protein S Agarose:

About 100 μl of a 50% suspension of S-protein agarose (Novagen) was incubated with or without 100 μg of int8 peptide with an S-protein tag, at room temperature for 1 hr, and then washed twice with 500 μl PBS. The agarose samples were then incubated at room temperature for 1 hr with 200 μg of transfected Cos-7 cell extract, then was washed twice with 500 μl of PBS with 1% NP40. The proteins associated with the resin were eluted at 92° C. for 2 min in 40 μl of SDS-sample buffer, and analyzed as a Western blot.

Growth assays. Cells (4-10⁴) were plated in quadruplicate in 24-well plates, incubated in serum-free DMEM for 24 hours, and treated with either 5 nM IGF-I (GroPep; Adelaide, Australia) or 10 mM HCl as vehicle. Following serum starvation, and for four subsequent days at 24-hour intervals, cell monolayers were washed with PBS and incubated for 30 minutes at 37° C. with 30 μl of MTS reagent [3-(4,5-dimethylthiazol-2-yl)-5-(3-carboxymethoxyphenyl)-2-(4-sulfophenyl-2H-tetrazolium) inner salt Aqueous One Solution (Promega; Madison, Wis.) dissolved in 270 ml PBS] per well. Absorbance readings were obtained at 490 nm in a Bio-Tek plate reader.

EGFR Inhibitor Studies

Control MCF7 cells were serum-starved overnight and treated with the EGFR kinase inhibitor AG1478 (conc. in DMSO) or vehicle for 5 min. prior to the addition of 14 nM EGF or 5 nM IGF-I. After 5 min. of growth factor treatment, cell lysates were prepared and analyzed for ERK and Akt/PKB activation as described above.

### EXAMPLE 2

### Herstatin, and its Intron-Encoded Receptor-Binding Domain, were Shown to Bind Specifically to IGF-1R with High (e.g., nm) Binding Affinity

The interaction of the receptor binding domain (RBD, encoded by HER-2 intron 8; int8 peptide) of herstatin with IGF-1R in transfected 3T3 cells was investigated. According to particular embodiments of the present invention, both full-length herstatin and its RBD bind specifically to IGF-1R with high binding affinity (e.g., nm), and IGF-IR was thus shown herein to be a target of herstatin.

Methods. Cell lines, expression vectors, protein purification, pull down assays, antibodies, Western blot analysis and ELISA assays were as described under EXAMPLE 1, herein above.

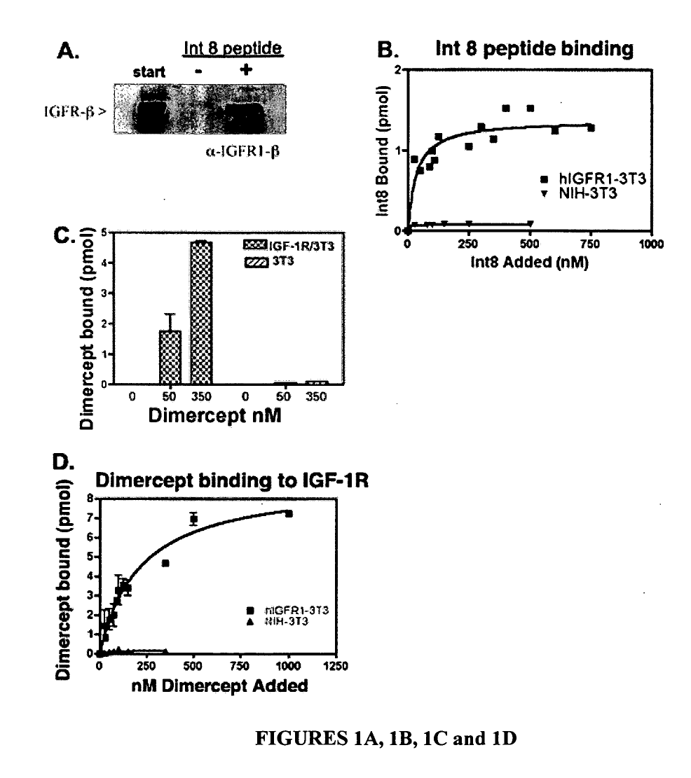

Results. FIG. 1A demonstrates that the Int8 peptide, purified from bacteria and immobilized on Protein S Sepharose™ ‘pulled down’ IGF-IR from 3T3 cell extracts, whereas Protein S Sepharose™ without peptide, or with an irrelevant peptide did not interact with IGF-IR.

Saturation binding of bacterial peptide Int8 to IGF-IR transfected 3T3 cells, and for comparison to parental 3T3 cells, was performed to determine the binding affinity of the Int8 peptide to IGF-1R. FIG. 1B shows saturable binding by the RBD Int8 polypeptide that is specific for IGF-IR. The Kd for binding, determined from this and other saturation binding curves was found to be in the nM range (e.g., in the 40 to 150 nM range), which is comparable to the binding affinity of Int8 peptide to HER-2 (Doherty et al., Supra) and to EGFR.

The interaction between full-length herstatin and IGF-1R was also investigated. FIG. IC shows that herstatin, purified from transfected S2 insect cells, exhibited dose-dependent binding to IGF-1R at nM concentrations.

FIG. 1D shows that full-length herstatin exhibited saturation binding to IGF-IR 3T3 cells, demonstrating nM binding affinity.

These results demonstrate that herstatin and its receptor binding domain bind specifically to IGF-1R with nM binding affinity (e.g., in the 40 to 150 nM range) and that IGF-IR is a target receptor of herstatin.

### EXAMPLE 3

### Herstatin was Shown to Prevent Activation of IGF-IR by IGF-1 in MCF7 Cells

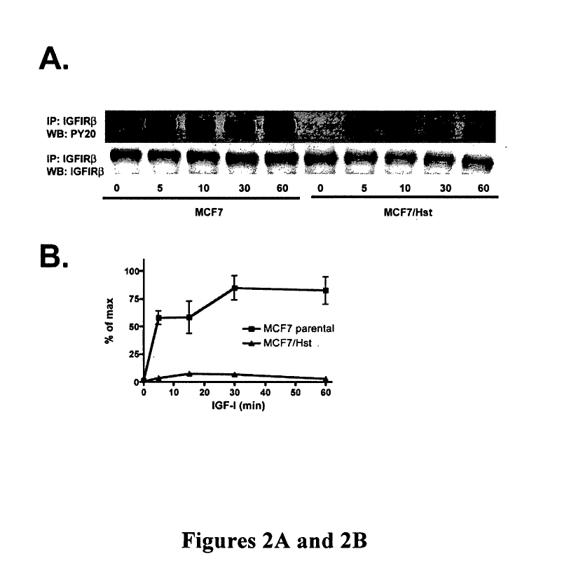

According to particular embodiments of the present invention, herstatin blocks activation of IGF-1R by IGF-1 (FIGS. 2A, 2B), and causes IGF-1R down-regulation (FIG. 2A, lower portion).

Methods. Cell lines, expression vectors, protein purification, pull down assays, antibodies, Western blot analysis and ELISA assays were as described under EXAMPLE I, herein above. IGF-I was added either to MCF-7 breast carcinoma cells, or to an MCF-7/herstatin cell line stably transfected with herstatin, to determine whether herstatin expression affects activation of the IGF-IR by its ligand. MCF7 and MCF7/Hst cells were serum-starved overnight, treated with 5 nM IGF-I over a 60-minute timecourse, and harvested in NP-40 lysis buffer. 1 mg of cell lysate was immunoprecipiated with IGF-IRβ antibody and protein A agarose beads. Immunoprecipitates were separated on a 10% SDS-PAGE gel and analyzed for IGF-IR expression and tyrosine phosphorylation. Western blots were scanned and quantified by densitometry.

Results. As expected, there is a robust IGF-I-mediated activation of the IGF-IR in MCF7 cells, demonstrated by enhanced tyrosine phosphorylated IGF-IR by 5 min (FIG. 2A, left panel). In contrast, activation of the IGF-IR by IGF (revealed by receptor tyrosine phosphorylation) was blocked in the herstatin-expressing MCF7 cells (FIG. 2A, right panel).

These results demonstrate that herstatin modulates IGF-IR-mediated signaling.

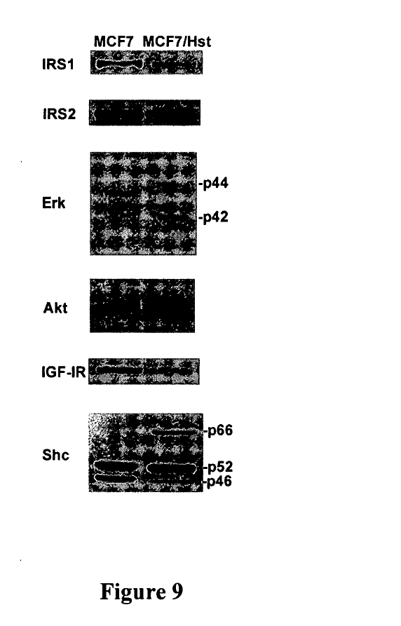

Additionally, as shown in FIG. 2A (lower portion), herstatin not only prevents activation of IGF-1R by IGF-1 in MCF-7 cells (upper panels), but also caused down-regulation of IGF-1R (lower panels). Likewise, herstatin-transfected MCF-7 cells show decreased expression of IRS-2 expression (also important in cell survival) when compared to non-transfected MCF-7 cells (FIG. 9).

### EXAMPLE 4

### The Herstatin RBD Int8 Polypeptide Bound in a Specific, Dose-Dependent Manner to EGFR, HER-2, HER-3, HER-4, IGF-1R and ΔEGFR, but did not Bind to a Mutant form of HER-3, FGFR-3, nor Mock-Transfected Cells

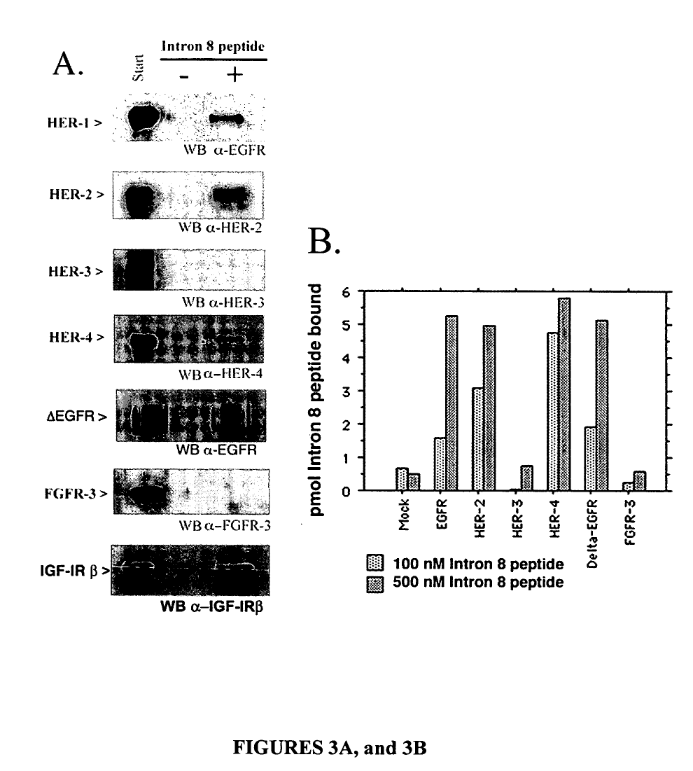

The binding of the intron 8-encoded RBD, expressed as a bacterial peptide (Int8) was investigated to identify other receptor targets of herstatin. The herstatin RBD Int8 polypeptide bound in a specific, dose-dependent manner to EGFR, HER-2, HER-3, HER-4, IGF-1R and ΔEGFR, but did not bind to a mutant form of HER-3, FGFR-3, or mock-transfected cells (FIGS. 3A and 3B).

Methods. Cell lines, expression vectors, protein purification, pull down assays, antibodies and ELISA assays were as described under EXAMPLE 1, herein above. Briefly, Protein S Sepharose™ with or without immobilized Int 8 peptide, was incubated with extracts from Cos 7 cells transiently transfected with several different receptors (or, in the case of IGF-1 with extracts from hIGFR-1-3T3 cells). Following washing steps, the protein bound to the agarose was analyzed as a Western blot with receptor-specific antibodies.

Results. As previously observed (Doherty et al., Supra.; Azios, Supra) EGFR and HER-2 from the transfected cell extracts bound specifically to the agarose with Int8 polypeptide (FIG. 3A). In particular assays, an Int8 peptide with the Arg to Ile mutation at residue 31 was somewhat less efficient in pulling-down the HER-2 receptor from the extracts (on average this herstatin variant appeared to bind about 2-fold less well that the comparable wild type sequence (SEQ ID NO:24)).

FIG. 3A also demonstrates that ΔEGFR, a tumor variant of the EGFR missing its N-terminal subdomains I and II (Nishikawa et al., Proc. Natl. Acad. Sci. USA 91:7727-31, 1994) specifically associated with Int8 polypeptide.

An additional member of the erbB family, HER-4, was also ‘pulled-down’ by Int8 agarose.

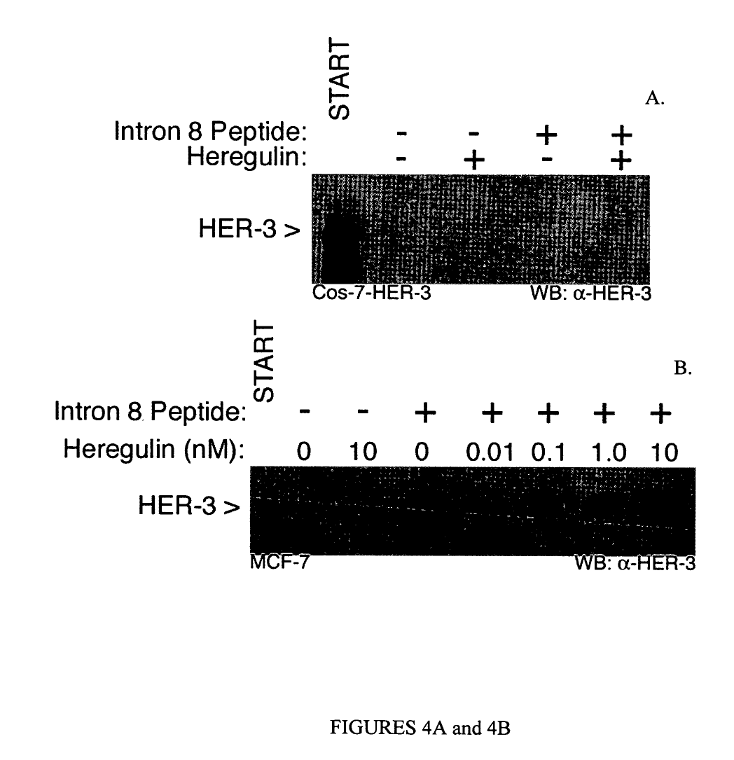

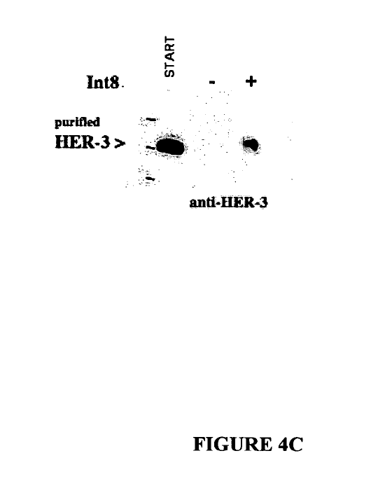

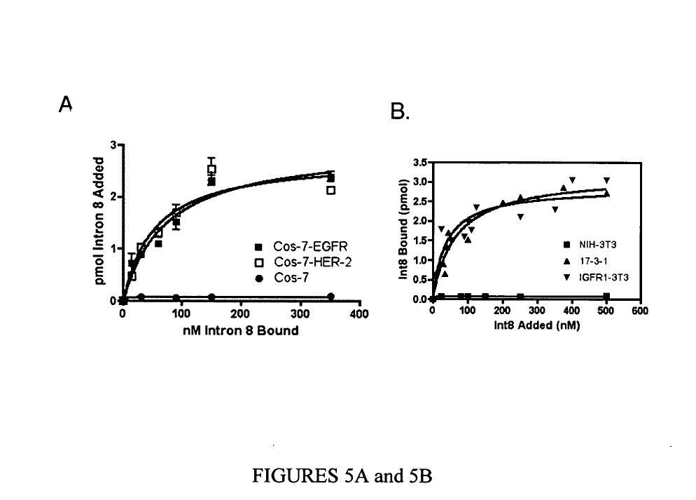

High-affinity binding by Int8 polypeptide to endogenous HER-3 in MCF7 breast cancer cells was observed, independent of ligand activation (FIG. 4B). Additionally, binding of the RBD Int8 polypeptide to purified (wild-type) HER-3 ectodomain expressed in stably transfected CHO cells was observed (FIG. 4C).

However, in the case of one particular form of HER-3 (corresponding to the product of the HER-3 expression vector, a gift from Dr. Tracy Ram, Washington State University in Pullman) there was no detectable association of the expressed HER-3 with Int8 polypeptide agarose, despite abundant expression in the respective transfected cells (FIG. 3A, third panel from top; and FIG. 4A). Applicants have determined that this non-Int8 binding form of HER-3 has a single point mutation resulting in substitution of Glu for Gly (relative to accession no.: NM_{—}001982, nucleotide # 1877, and amino acid residue position 560) in the ectodomain of HER-3.

As disclosed in EXAMPLE 2 above with respect to the interaction of the Int8 polypeptide with the IGF-1R, specific ‘pull-down’ of the β subunit of the IGF-IR from transfected cell extracts was observed (FIG. 3A, bottom panel). This result may reflect the fact that the IGF-1R contains regions of ectodomain sequence homology with the EGFR (Garrett et al., Cell 110:763-73, 2002).

The FGFR-3, a receptor tyrosine kinase with Ig-like motifs and no structural homology with the ErbB family ectodomains, did not bind to the Int8 peptide (FIG. 3A).

Therefore, according to particular aspects of the present invention, the herstatin RBD Int8 polypeptide binds in a high-affinity, specific manner to EGFR, HER-2, HER-3. HER-4, IGF-1R and ΔEGFR, but does not bind to a mutant form of HER-3 (single point mutation resulting in substitution of Glu for Gly at amino acid position 560), to FGFR-3, or to mock-transfected cells.

ELISA assay results. ELISA analysis used to quantify bound RBD Int8 polypeptide to further examine interaction of the int8 polypeptide with the extracellular domain of the various receptors at the cell surface. As was shown for the IGF-1R (hIGF-1R-3T3 cells) in EXAMPLE 1 above, FIG. 3B shows that the Int8 polypeptide bound in a specific and dose-dependent manner to EGFR, HER-2, HER-4, and ΔEGFR, but not to a mutant form of HER-3, FGFR-3, or mock-transfected Cos-7 cells, in agreement with results obtained by the ‘pull-down’ assays FIG. 3A).

Binding affinities were further characterized by generating saturation-binding curves (FIGS. 5A and 5B). The RBD Int8 polypeptide bound with high affinity to HER-2-transfected Cos-7 cells (in particular assays, K_{D}=50±6 nM; FIG. 5A, open squares; among various assays, in the 40 to 150 nM range) and to EGFR-transfected Cos-7 cells (in particular K_{D}=78±10 nM; FIG. 5A, filled squares; among various assays in the 40 to 150 nM range) with binding affinities, assessed by comparative nonlinear regression analysis, that were not significantly different (P=0.40) (FIG. 5A). Furthermore, similar to the determination of EXAMPLE 2 above (K_{D}=40nM in particular assays; among various assays in the 40 to 150 nM range), the RBD Int8 polypeptide bound to the IGF-IR/3T3 cells with an affinity (K_{D}=70±21 in particular assays; among various assays in the 40 to 150 nM range) that was not significantly different (P=0.96) from the affinity for HER-2/3T3 cells (K_{D}=66±16) (FIG. 5B) (among various assays in the 40 to 150 nM range).

In particular assays, the mutant Int8 polypeptide with Arg31I1e bound somewhat less well (perhaps 2-fold) to the HER-2 receptor overexpressing cells, even though the herstatin ELISA detected the wildtype and mutant peptide equally.

These results show, therefore, that the RBD Int8 polypeptide bound to EGFR, HER-2, and IGF-1R with similar (overlapping) binding affinities.

### EXAMPLE 5

### Relative Binding of Herstatin Between 3T3/HER-2 and 3T3/IGF-IR Cells, and Between 3T3/HER-2 and Cos-7/EGFcells was Directly Compared, and the Relative Affinities of Herstatin and RGB Int8 Polypeptide were Determined on 3T3/HER-2 Cells

ELISA analysis was performed to compare relative binding of herstatin between 3T3/HER-2 and 3T3/IGF-IR cells, and between 3T3/HER-2 and Cos-7/EGFcells. Additionally, the relative affinities of herstatin and RGB Int8 polypeptide were determined on 3T3/HER-2 cells.

Methods. Cell lines, expression vectors, protein purification, antibodies and ELISA assays were as described under EXAMPLE 1, herein above.

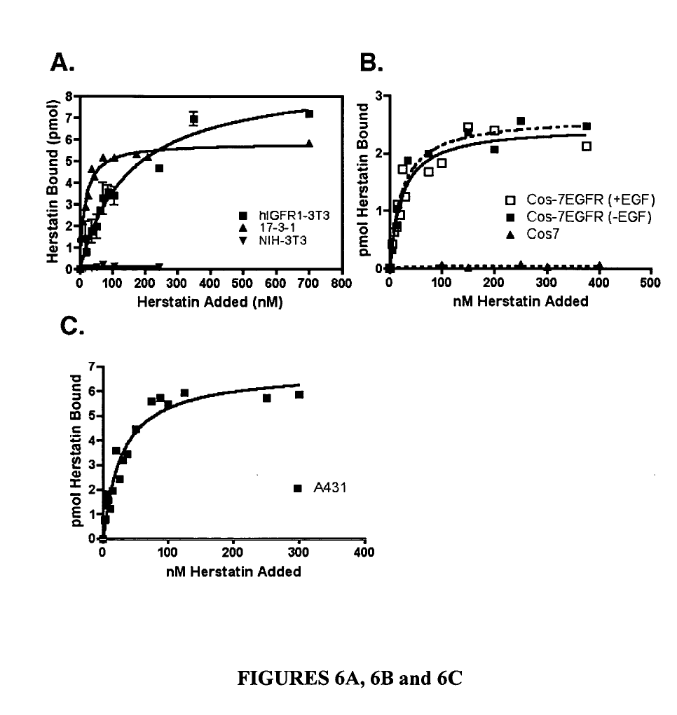

Results. A direct comparison of the binding of herstatin to 3T3/HER-2 and 3T3/IGF-IR cells revealed that the affinity for the IGF-1R (K_{D} ˜151 nM) was lower (P<0.0001) by about 10-fold (FIG. 6A). The full-length herstatin bound to 3T3/HER-2 cells with a K_{D}=14.7±1.8 nM, which is greater than the binding affinity of RBD Int8 polypeptide (P<0.0001) by 3-4 fold (FIG. 6A).

The dissociation constant of FIG. 6A for EGFR was similar to that of HER-2, and was unaffected by ligand occupation indicated by a K_{D}=16.4±3.6 nM versus 16.3±3.6 nM (respectively) for Cos-7/EGFR treated or not with 10 nM EGF (FIG. 6B).

### EXAMPLE 6

### Herstatin Exhibited Saturation Binding to Endogenous Receptors in A431 Epidermoid Carcinoma Cells

Herstatin binding to endogenous receptors in A431 epidermoid carcinoma cells was investigated to determine if a one-affinity site binding model was the best fit for EGFR-specific binding of herstatin, in the presence and absence of EGF.

Methods. A431 cells were from ATCC.

Results. Herstatin exhibited saturation binding to endogenous receptors in A431 epidermoid carcinoma cells, which express very high levels of EGFR and low levels of other ErbB receptors (FIG. 6C). At saturation, 6.9±0.4 pmol of herstatin were bound indicating about 2×10⁶ binding sites/cell, which matches the number of EGFR per A431 cell at 2×10⁶ (Filmus et al., Biochem. Biophys. Res. Commun., 131:207-15, 1985; Filmus et al., Biochem. Biophys. Res. Commun. 128:898-905, 1985). Comparison of nonlinear models indicated that a hyperbolic one-affinity site binding model was the best fit for EGFR-specific binding of herstatin, in the presence and absence of EGF.

### EXAMPLE 7

### Herstatin Effects were Shown to be Receptor Specific

Because herstatin binds to multiple receptors, binding studies were performed to demonstrate that the effects of herstatin are receptor-specific.

Methods. Cells and western blot analysis were as described under EXAMPLE 1 above.

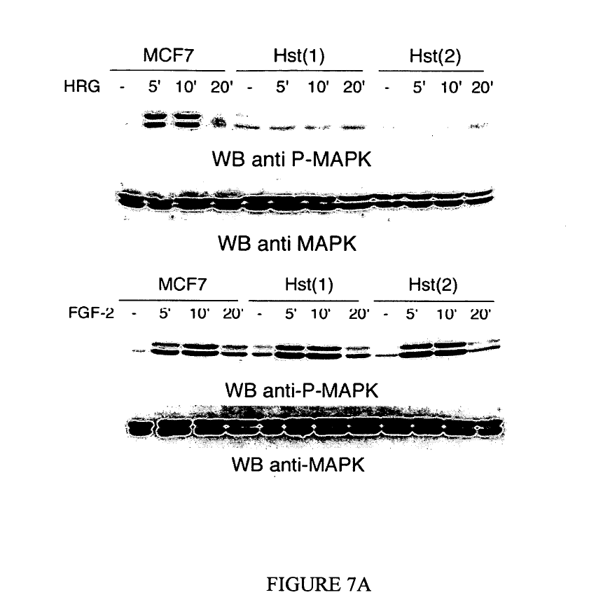

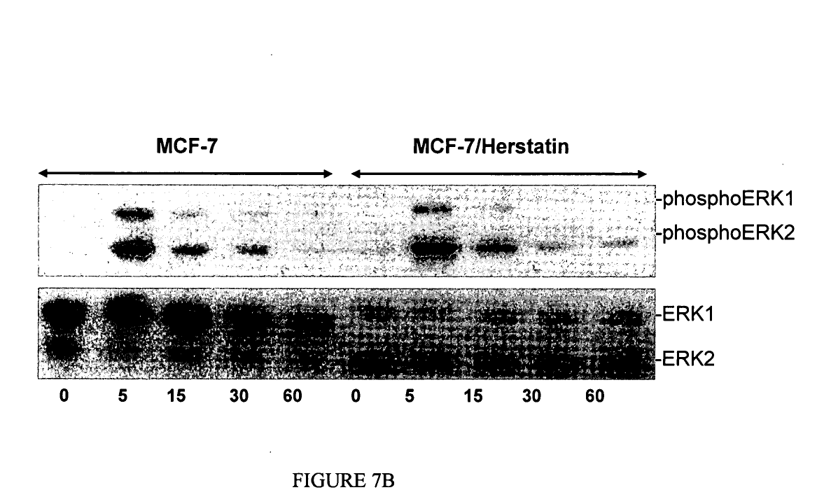

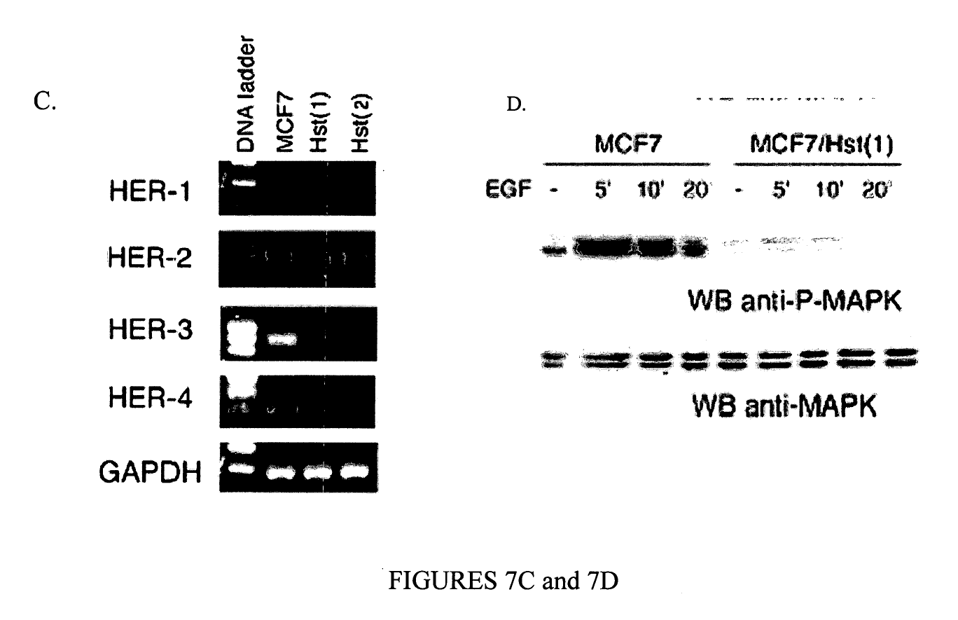

Results. As demonstrated herein above, herstatin does not bind to the FGFR. FIG. 7A (upper panel) and FIG. 7D show that herstatin blocks intracellular signaling (MAPK phosphorylation) by Heregulin (the ligand for HER-3 and HER-4) and EGF (the ligand for the EGFR), respectively, in MCF-7 cells, whereas herstatin does not affect FGF signaling (MAPK phosphorylation) in MCF-7 cells (FIG. 7A, lower panel), and does not inhibit IGF-1-mediated ERK phosphorylation in MCF-7 cells (FIG. 7B).

Additionally, FIG. 7C shows that herstatin down-regulates HER-1, HER-3 and HER-4 receptors in MCF-7 cells.

### EXAMPLE 8

### Herstatin Inhibited Heregulin/HER-4-Mediated Activation of, and IGF-1/IGF1R-Mediated Activation of the PI3/Akt Pathway that is Important in Cell Survival

The physiological effects of herstatin on HER-4-mediated signaling were investigated. The protein kinase called Akt is a key regulator of cellular survival. Activation of Akt is both necessary and sufficient for survival of cells. Stimulation of activated Akt causes inappropriate cell survival, or prevents normal cell death, which has been found to occur in several human cancers. HER-2 and the EGF receptor, for example, both cause activation of the Akt survival signal whereby, according to current theory and belief, they cause oncogenic growth (Blume-Jensen & Hunter, Nature 411:355-365, 2001; Datta et al., Genes and Development 13:2905-2907, 2000; and Yarden & Slikowski, Nature Reviews, Molecular Cell Biology, 2:127-137).

Methods. Measurement of activated phospho-akt (activated AKT) in EGFR3T3 cells. Measurement of activated AKT (phospho-akt) was accomplished using standard Western blotting techniques, employing a commercially available anti-phospho-akt antibody (Santa Cruz). Briefly, CHO cells were transfected with HER-4 alone, or cotransfected with HER-4 and and herstatin. Twenty-four (24) hours after transfection, serum-starved cells were treated with heregulin or vehicle for 15 and 30 min. The cells were extracted and analyzed as a Western blot with antibodies specific for activated Akt (anti-phospho-Akt), or for total Akt.

Results. Heregulin caused a robust increase in phospho-Akt in the absence of herstatin, whereas heregulin induction of phosphoAkt was reduced in herstatin expressing cells.

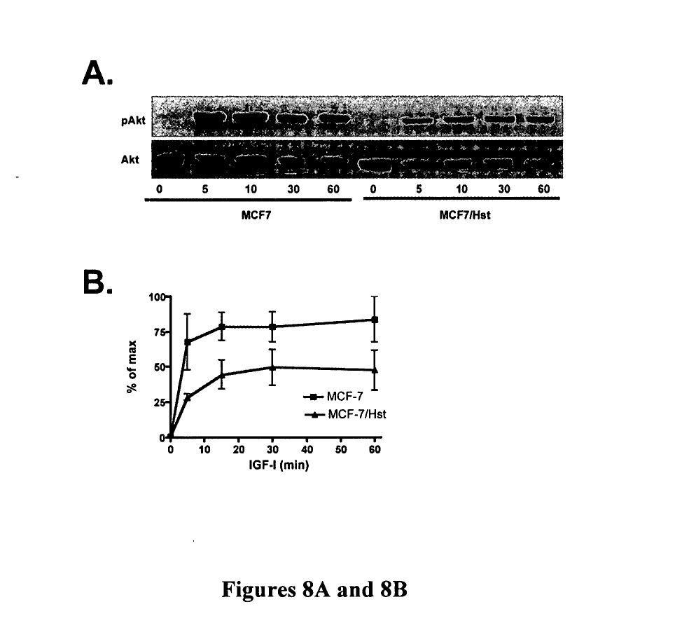

Additionally, as shown in FIG. 8, herstatin inhibited IGF-1/IGF-1R-mediated activation of the PI3/Akt pathway.

Furthermore, FIG. 9 shows the effect of herstatin-expression on the expression levels of various signaling proteins. Herstatin expression in MCF7 breast carcinoma cells down-regulated IGF-1R, IRS-1, IRS-2 (also important in cell survival), and pKB/Akt expression, but MAPK expression was unaffected. Herstatin expression also induced expression of the p66 isoform of Shc, which is not detectable by Western Blot in parental MCF7 cells.

Therefore, according to particular aspects of the present invention, herstatin inhibits activation of the PI3/Akt and IRS-2 pathways that are important in cell survival.

### EXAMPLE 9

### Herstatin Inhibited IGF-1-Mediated Survival of MCF7 Cells

Previous studies have shown that stable expression of herstatin in MCF7 breast carcinoma cells resulted in diminished heregulin-stimulated proliferation (Jhabvala-Romero et al., Oncogene 22:8178-86, 2003). To further investigate the effect of herstatin on IGF-I action, the IGF-I-induced growth of parental MCF7 cells and two clones stably transfected with herstatin (MCF7/Hst#1 and MCF7/Hst#2) was investigted.

Methods. MCF7 parental and MCF7/Hst breast cancer cells, stably transfected with herstatin (either MCF7/Hst#1, a low-level herstatin-expressing clone, or MCF7/Hst#2, a relatively high-level herstatin-expressing clone), were plated into 24-well plates at 40,000 cells per well overnight and the MTS assay was conducted in triplicate wells to quantify viable cells at time zero. The cells were then treated in serum-free media with vehicle or with 10 nM IGF-I and triplicate wells were quantified by the MTS assay on day 1, 2 and 3. The results are plotted as mean percent of the start at zero time. The error bars represent the standard error of the mean.

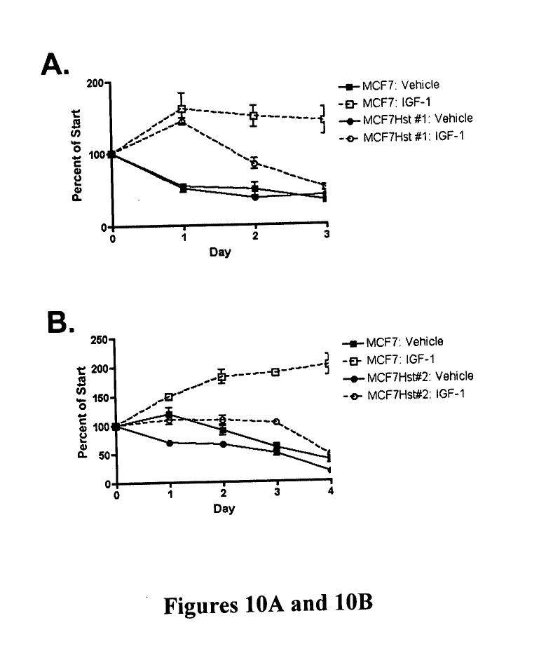

Results. FIGS. 10A and 10B show that herstatin expression blocks IGF-1-mediated survival of MCF7 cells. Parental MCF7 cells grew in response to IGF-I, whereas cell viability decreased in the absence of growth factor. Both of the MCF7/Hst clones, however, failed to exhibit IGF-I-stimulated growth. Furthermore, the growth reduction occurred faster in clone #1, which expresses relatively more herstatin, indicating that herstatin affects IGF-I-mediated growth in a concentration-dependent manner.

### PARTICULAR REFERENCES CITED

Olayioye et al., Embo J 19:3159-67, 2000.
Dougall et al., Oncogene 9:2109-23, 1994.
Hynes & Stem, Biochim Biophys Acta 1198:165-84, 1994.
Tzahar & Yarden, Biochim Biophys Acta 1377:M25-37, 1998.
Doherty et al., Proc Natl Acad Sci U S A 96:10869-74, 1999.
Azios et al., Oncogene 20:5199-209, 2001.
Jhabvala-Romero et al., Oncogene 22:8178-86, 2003.
Justman & Clinton, J Biol Chem. 277:20618-24, 2002.
Chakravarti et al., Cancer Res. 62:200-7, 2002.
Lu et al., J Biol Chem. 279:2856-65, 2004.
Lu et al., J Natl Cancer Inst. 93:1852-7, 2001.
Baserga, R. L., Hum Pathol. 31:275-6, 2000.
Wang & Sun, Curr Cancer Drug Targets 2:191-207, 2002.
Lin et al., Mol Cell Endocrinol. 69:111-9, 1990.
Christianson et al., Cancer Res. 58:5123-9, 1998.
Nishikawa et al., Proc Natl Acad Sci U S A 91:7727-31, 1994.
Garrett et al., Cell 110:763-73, 2002.
Filmus et al., Biochem Biophys Res Commun. 131:207-15, 1985.
Filmus et al., Biochem Biophys Res Commun. 128:898-905, 1985.

1. A method for treating a condition characterized by altered cellular receptor expression or receptor-mediated signaling, comprising administering to a subject in need thereof, a therapeutically effective amount of a herstatin, or of a variant thereof, that binds to the extracellular domain of at least one target receptor of a target cell of the subject, wherein the at least one target receptor is selected from the group consisting of: ΔEGFR; HER-3 (erbB-3); HER-4 (erbB-4) and IGF-1:

2. The method of claim 1, wherein the condition is a cellular proliferative condition or disorder.

3. The method of claim 2, wherein the cellular proliferative condition or disorder is cancer.

4. The method of claim 1, wherein the target cell does not express EGFR (HER-1, erbB-1) or HER-2 (erbB-2), or does not express either.

5. The method of claim 3, wherein the cancer is selected from the group consisting of breast cancer, gastric cancer, colon, lung cancer, glioblastoma ovarian cancer, pancreatic cancer and prostate cancer.

6. The method of claim 1, wherein the altered cellular receptor expression or receptor-mediated signaling is that of a receptor selected from the group consisting of EGFR (HER-1, erbB-1); ΔEGFR; HER-2 (erbB-2); HER-3 (erbB-3); HER-4 (erbB-4) and IGF-1.

7. The method of claim 3, wherein the cancer is refractory, at least to some extent, to treatment by at least one other therapeutic agent that is specific for a receptor selected from the group consisting of: EGFR (HER-1, erbB-1); ΔEGFR; HER-2 (erbB-2); HER-3 (erbB-3); HER-4 (erbB-4) and IGF-1, and wherein the at least one other therapeutic agent is different from herstatin, herstatin variants, int8 RDB polypeptides, and int8 RDB polypeptide variants.

8. The method of claim 7, wherein the cancer is breast cancer, or prostate cancer.

9. The method of claim 7, wherein the at least one other agent comprises a receptor-specific antibody, or a small-molecule receptor tyrosine kinase inhibitor.

10. The method of claim 9, wherein at least one other agent is the HER-2-specific antibody rhuMAb4D5.

11. The method of claim 1, wherein the herstatin, or variant thereof, comprises a polypeptide selected from the group consisting of SEQ ID NO:2, or a fragment of SEQ ID NO:2 of about 80 to 419 contiguous residues in length, including the C-terminal 79 contiguous amino acids of SEQ ID NO:2.

12. The method of claim 11, wherein the herstatin, or variant thereof binds to the extracellular domain of the at least one target receptor with an affinity binding constant of at least 10⁷ M⁻¹.

13. The method of claim 1, further comprising administering a therapeutically effective amount of a receptor-specific antibody that binds to the extracellular domain of a cellular receptor of the target cell.

14. The method of claim 13, wherein the receptor-specific antibody binds to a cellular receptor selected from the group consisting of: EGFR (HER-1, erbB-1); ΔEGFR; HER-2 (erbB-2); HER-3 (erbB-3); HER-4 (erbB-4) and IGF-1.

15. The method of claim 14, wherein the receptor-specific antibody is the HER-2-specific antibody rhuMAb4D5.

16. The method of claim 13, wherein the receptor-specific antibody binds to a cellular receptor of the target cell that is different from the at least one cellular receptor bound by the herstatin, or the variant thereof.

17. The method of claim 3, further comprising administration of a therapeutically effective amount of a chemotherapeutic agent.

18. The method of claim 17, wherein the chemotherapeutic agent is an anti-neoplastic agent selected from the group consisting of: cyclophosphamide, triethylenephosphoramide, triethylenethiophosphoramide, flutamide, altretamine, triethylenemelamine, trimethylolmelamine, meturedepa, uredepa, aminoglutethimide, L-asparaginase, BCNU, benzodepa, bleomycin, busulfan, camptothecin, capecitabine, carboquone, chlorambucil, cytarabine, dactinomycin, daunomycin, daunorubicin, docetaxol, doxorubicin, epirubicin, estramustine, dacarbazine, etoposide, fluorouracil, gemcitabine, hydroxyurea, ifosfamide, improsulfan, mercaptopurine, methotrexate, mitomycin, mitotane, mitoxantrone, novembrichin, paclitaxel, piposulfan, plicamycin, prednimustine, procarbazine, tamoxifen, temozolomide, teniposide, thioguanine, thiotepa, UFT, uracil mustard, vinblastine, vincristine, vinorelbine and vindesine.

19. The method of claim 1, wherein the herstatin, or variant thereof, comprises SEQ ID NO:23.

20. A method for treating a condition characterized by altered cellular receptor expression or receptor-mediated signaling, comprising administering to a subject in need thereof, a therapeutically effective amount of an Int8 RBD polypeptide, or a variant thereof, that binds to the extracellular domain of at least one target receptor of a target cell of the subject, wherein the at least one target receptor is selected from the group consisting of: ΔEGFR; HER-3 (erbB-3); HER-4 (erbB-4) and IGF-1.

21. The method of claim 20, wherein the condition is a cellular proliferative condition or disorder.

22. The method of claim 21, wherein the cellular proliferative condition or disorder is cancer.

23. The method of claim 20, wherein the target cell does not express EGFR (HER-1, erbB-1) or HER-2 (erbB-2), or does not express either.

24. The method of claim 22, wherein the cancer is selected from the group consisting of breast cancer, gastric cancer, colon, lung cancer, glioblastoma ovarian cancer, pancreatic cancer and prostate cancer.

25. The method of claim 20, wherein the altered cellular receptor expression or receptor-mediated signaling is that of a receptor selected from the group consisting of EGFR (HER-1, erbB-1); ΔEGFR; HER-2 (erbB-2); HER-3 (erbB-3); HER-4 (erbB-4) and IGF-1.

26. The method of claim 22, wherein the cancer is refractory, at least to some extent, to treatment by at least one other therapeutic agent that is specific for a receptor selected from the group consisting of: EGFR (HER-1, erbB-1); ΔEGFR; HER-2 (erbB-2); HER-3 (erbB-3); HER-4 (erbB-4) and IGF-1, and wherein the at least one other therapeutic agent is different from herstatin, herstatin variants, int8 RDB polypeptides, and int8 RDB polypeptide variants.

27. The method of claim 26, wherein the cancer is breast cancer, or prostate cancer.

28. The method of claim 26, wherein the at least one other agent comprises a receptor-specific antibody, or a small-molecule receptor tyrosine kinase inhibitor.

29. The method of claim 26, wherein at least one other agent is the HER-2-specific antibody rhuMAb4D5.

30. The method of claim 20, wherein the Int8 RBD polypeptide, or a variant thereof, comprises a polypeptide selected from the group consisting of SEQ ID NO:1, or a fragment of SEQ ID NO:1 of about 50 to 79 contiguous residues in length.

31. The method of claim 30, wherein the Int8 RBD polypeptide, or a variant thereof binds to the extracellular domain of the at least one target receptor with an affinity binding constant of at least 10⁷ M⁻¹.

32. The method of claim 20, further comprising administering a therapeutically effective amount of a receptor-specific antibody that binds to the extracellular domain of a cellular receptor of the target cell.

33. The method of claim 32, wherein the receptor-specific antibody binds to a cellular receptor selected from the group consisting of: EGFR (HER-1, erbB-1); ΔEGFR; HER-2 (erbB-2); HER-3 (erbB-3); HER-4 (erbB-4) and IGF-1.

34. The method of claim 33, wherein the receptor-specific antibody is the HER-2-specific antibody rhuMAb4D5.

35. The method of claim 32, wherein the receptor-specific antibody binds to a cellular receptor of the target cell that is different from the at least one cellular receptor bound by the Int8 RBD polypeptide, or the variant thereof.

36. The method of claim 22, further comprising administration of a therapeutically effective amount of a chemotherapeutic agent.

37. The method of claim 36, wherein the chemotherapeutic agent is an anti-neoplastic agent selected from the group consisting of: cyclophosphamide, triethylenephosphoramide, triethylenethiophosphoramide, flutamide, altretamine, triethylenemelamine, trimethylolmelamine, meturedepa, uredepa, aminoglutethimide, L-asparaginase, BCNU, benzodepa, bleomycin, busulfan, camptothecin, capecitabine, carboquone, chlorambucil, cytarabine, dactinomycin, daunomycin, daunorubicin, docetaxol, doxorubicin, epirubicin, estramustine, dacarbazine, etoposide, fluorouracil, gemcitabine, hydroxyurea, ifosfamide, improsulfan, mercaptopurine, methotrexate, mitomycin, mitotane, mitoxantrone, novembrichin, paclitaxel, piposulfan, plicamycin, prednimustine, procarbazine, tamoxifen, temozolomide, teniposide, thioguanine, thiotepa, UFT, uracil mustard, vinblastine, vincristine, vinorelbine and vindesine.

38. The method of claim 20, wherein the Int8 RBD polypeptide, or variant thereof, comprises SEQ ID NO:24.

39. A method for targeting a therapeutic agent to target cells, comprising attaching the therapeutic agent to herstatin, or to a variant thereof, that binds to the extracellular domain of at least one target receptor of a target cell being targeted, wherein the at least one target receptor is selected from the group consisting of: ΔEGFR; HER-3 (erbB-3); HER-4 (erbB-4) and IGF-1.

40. The method of claim 39, wherein the target cell is a cancer cell.

41. The method of claim 39, wherein the target cell does not express EGFR (HER-1, erbB-1) or HER-2 (erbB-2), or does not express either.

42. The method of claim 40, wherein the cancer is selected from the group consisting of breast cancer, gastric cancer, colon, lung cancer, glioblastoma ovarian cancer, pancreatic cancer and prostate cancer.

43. The method of claim 39, wherein the herstatin, or variant thereof, comprises a polypeptide selected from the group consisting of SEQ ID NO:2, or a fragment of SEQ ID NO:2 of about 80 to 419 contiguous residues in length, including the C-terminal 79 contiguous amino acids of SEQ ID NO:2.

44. The method of claim 43, wherein the herstatin, or variant thereof binds to the extracellular domain of the at least one target receptor with an affinity binding constant of at least 10⁷ M⁻¹.

45. The method of claim 39, wherein the herstatin, or variant thereof, comprises SEQ ID NO:23.

46. A method for targeting a therapeutic agent to target cells, comprising attaching the therapeutic agent to an Int8 RBD polypeptide, or to a variant thereof, that binds to the extracellular domain of at least one target receptor of a target cell being targeted, wherein the at least one target receptor is selected from the group consisting of: ΔEGFR; HER-3 (erbB-3); HER-4 (erbB-4) and IGF-1.

47. The method of claim 46, wherein the target cell is a cancer cell.

48. The method of claim 46, wherein the target cell does not express EGFR (HER-1, erbB-1) or HER-2 (erbB-2), or does not express either.

49. The method of claim 47, wherein the cancer is selected from the group consisting of breast cancer, gastric cancer, colon, lung cancer, glioblastoma ovarian cancer, pancreatic cancer and prostate cancer.

50. The method of claim 46, wherein the Int8 RBD polypeptide, or a variant thereof, comprises a polypeptide selected from the group consisting of SEQ ID NO:1, or a fragment of SEQ ID NO:1 of about 50 to 79 contiguous residues in length.

51. The method of claim 50, wherein the Int8 RBD polypeptide, or a variant thereof binds to the extracellular domain of the at least one target receptor with an affinity binding constant of at least 10⁷ M⁻¹.

52. The method of claim 46, wherein the Int8 RBD polypeptide, or variant thereof, comprises SEQ ID NO:24.

53. A pharmaceutical composition for treating a condition characterized by altered cellular receptor expression or receptor-mediated signaling, comprising, along with a pharmaceutically acceptable carrier or excipient, a first agent selected from the group consisting of: herstatin, or a variant thereof; a Int8 RBD polypeptide, or a variant thereof; and combinations thereof, the composition further comprising a second agent selected from the group consisting of: a receptor-specific antibody that binds to the extracellular domain (ECD) of a cellular receptor of the target cell; a small molecule receptor tyrosine kinase inhibitor; and combinations thereof, with the proviso that the receptor-specific antibody is not a HER-1 or HER-2-specific antibody.

54. The composition of claim 53, wherein the receptor-specific antibody is a therapeutic antibody.

55. The composition of claim 53, wherein the receptor-specific antibody binds to a cellular receptor selected from the group consisting of: ΔEGFR; HER-3 (erbB-3); HER-4 (erbB-4) and IGF-1.

56. The composition of claim 53, wherein the herstatin, or variant thereof, comprises a polypeptide selected from the group consisting of SEQ ID NO:2, or a fragment of SEQ ID NO:2 of about 80 to 419 contiguous residues in length, including the C-terminal 79 contiguous amino acids of SEQ ID NO:2.

57. The composition of claim 53, wherein the Int8 RBD polypeptide, or a variant thereof comprises a polypeptide selected from the group consisting of SEQ ID NO:1, or a fragment of SEQ ID NO:1 of about 50 to 79 contiguous residues in length.

58. The composition of claim 53, wherein the herstatin, or variant thereof, comprises SEQ ID NO:23.

59. The composition of claim 53, wherein the Int8 RBD polypeptide, or variant thereof, comprises SEQ ID NO:24.

60. A method for identification of cells having HER-3 receptors that do not bind herstatin, int 8 RDB polypeptides, or variants thereof, comprising: obtaining a cellular sample; and determining, using one or more suitable assays, whether the cells express SEQ ID NO:14, wherein cells having HER-3 receptors that do not bind herstatin are identified if SEQ ID NO:14 is expressed.

61. A method for screening for cells that are, at least to some extent, non-responsive to herstatin, int 8 RDB polypeptides, or variants thereof, comprising obtaining a cellular sample; and determining, using one or more suitable assays, whether the cells express SEQ ID NO:14, wherein the cells are determined to be, at least to some extent, non-responsive to herstatin, int 8 RDB polypeptes, or variants thereof, if the cells express SEQ ID NO:14.
